(19) United States
(12) Patent Application Publication
(10) Pub. No.: US 2026/0216159 A1
(43) Pub. Date: Jul. 30, 2026

(54) METHOD OF PREPARING TRANSDERMAL DELIVERY SYSTEM

(71) Applicant: Corium, LLC, Cambridge, MA (US)

(72) Inventor: Eun Soo LEE, Redwood City, CA (US)

(21) Appl. No.: 19/142,732

(22) PCT Filed: Dec. 27, 2023
(86) PCT No.: PCT/US2023/085970

§ 371 Date: Jun. 24, 2025

Related U.S. Application Data
(60) Provisional application No. 63/477,434, filed on Dec. 28, 2022.
(60) Provisional application No. 63/589,774, filed on Oct. 12, 2023.

(51) Int. Cl. A61K 31/445; A61K 9/00; A61K 9/70; A61K 47/02; A61K 47/10; A61K 47/14; A61K 47/32

(52) U.S. Cl. CPC A61K 31/445; A61K 9/0014; A61K 9/7007; A61K 47/02; A61K 47/10; A61K 47/14; A61K 47/32

(57) ABSTRACT

The present disclosure provides a method of preparing a drug matrix layer and transdermal delivery systems for delivering donepezil free base to patients suffering from central nervous system disorders including dementia and Alzheimer's.

### CROSS-REFERENCES TO RELATED APPLICATIONS

This application claims priority to U.S. Provisional Application Nos. 63/589,774, filed Oct. 12, 2023, and 63/477,434, filed Dec. 28, 2022, each of which is incorporated herein in its entirety for all purposes.

### BACKGROUND

Transdermal drug delivery systems can be an effective means for administering active pharmaceutical agents that might have disadvantages when administered via other routes such as orally or parenterally. However, the delivery of many drugs over a long period of time (e.g. several days or more) is difficult. Transdermal delivery of basic (i.e., alkaline) drugs can be especially difficult due to poor skin permeability. Further, some active agents have poor or low solubility in the adhesive and/or other components used in typical transdermal formulations. Further, there is a need for stable, long term administration of active agents (e.g. 1-10 days or more) that provides a stable and effective release of the agent over the administration period and has suitable adhesion for the long term administration.

Active agents for transdermal delivery are typically provided in their neutral form because the neutral form is typically much more skin permeable than a corresponding salt form. In traditional transdermal formulations, a neutral form of an active agent is solubilized in an adhesive matrix, and the active agent diffuses through the adhesive matrix and into the skin. Transdermal patches, therefore, typically contain as much active agent dissolved in the adhesive matrix as the agent's solubility in the adhesive matrix allows, often with solubilizers to enhance its solubility. Alternatively, neutral, solid particles of active agent are sometimes dispersed in an adhesive matrix, so long as the particles' dissolution rate is such that a constant supply of dissolved active agent is provided.

For many active agents, however, a neutral form is more difficult to solubilize and/or formulate into a composition, system or medicament for administration to a subject. When a drug has a low solubility in an adhesive matrix, as does a non-ionized neutral form, it is difficult to incorporate a sufficient amount of the drug in a solubilized form in the adhesive in order to deliver at a therapeutic level for multiple days. A further complication is that a dissolved active agent may crystallize within the adhesive matrix during the process of preparing the medicament, e.g., solvation, coating, and drying. Further, many active agents are less stable in neutral form than in salt form. Other challenges for transdermal patches can include delamination of the backing layer. Therefore, there exists a need for compositions, systems and medicaments having an adhesive matrix as a component layer that can consistently and effectively deliver a therapeutic amount of an active agent over a prolonged period of time. There also exists a need for transdermal patches with improved adhesion between the backing layer and the the remainder of the patch to reduce delamination of the backing layer.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY OF THE INVENTION

In one embodiment, the present invention provides a method of preparing a drug matrix layer, comprising: forming a first mixture comprising donepezil HCl polymorph Form III, and sodium bicarbonate, thereby preparing the drug matrix layer.

In another embodiment, the present invention provides a drug matrix layer prepared by the method of the present invention.

In another embodiment, the present invention provides a method for preparing a transdermal delivery system, comprising:
(i) laminating a microporous membrane layer onto a top surface of a contact adhesive layer to form a contact adhesive laminate having a top surface and a bottom surface;
(ii) preparing a drug matrix layer comprising:
   forming a first mixture comprising triethyl citrate, lauryl lactate, and ethyl acetate,
   adding ascorbyl palmitate to the first mixture,
   adding polyvinylpyrrolidone to the first mixture,
   adding donepezil HCl polymorph Form III to the first mixture,
   adding sorbitan monolaurate to the first mixture,
   adding sodium bicarbonate and glycerin to the first mixture, wherein the sodium bicarbonate is present in a molar ratio of at least 0.85 to the donepezil HCl,
   adding an acrylate polymer to the first mixture,
   coating the first mixture on a release liner,
   drying the coated mixture, and
   removing the release liner, thereby preparing the drug matrix layer;
(iii) laminating the drug matrix layer onto the top surface of the contact adhesive laminate to form a drug matrix laminate having a top surface and a bottom surface;
(iv) laminating a separating layer onto the top surface of the drug matrix laminate to form an active laminate having a top surface and a bottom surface, wherein the separating layer comprises a top surface and a bottom surface, wherein the top surface of the separating layer comprises a coating of ethylene-vinyl acetate copolymer, and wherein the bottom surface of the separating layer is in contact with the top surface of the drug matrix laminate;
(v) laminating a polyester fabric onto an adhesive overlay layer comprising acrylate polymer to form a backing layer having a top surface and a bottom surface;
(vi) laminating the bottom surface of the backing layer onto the top surface of the active laminate so that the adhesive overlay layer is in contact with the top surface of the active laminate;
(vii) treating the top surface of the separating layer with a corona discharge treatment to form a treated separating layer,
   wherein the corona discharge treatment is performed using a power of from 0.10 kW to 0.12 kW and a power density of from 2.1 to 2.6 W/ft²/min,
   wherein the treated separating layer comprises a top surface and a bottom surface such that the top surface of the treated separating layer has a surface energy of at least 40 Dynes, and
   wherein the bottom surface of the contact adhesive layer is in contact with a first process liner;
(viii) removing the first process liner to expose the bottom surface of the contact adhesive layer; and
(ix) laminating a release liner onto the bottom surface of the contact adhesive layer, thereby forming the transdermal delivery system.

In another embodiment, the present invention provides a transdermal delivery system prepared by the method of the present invention.

### BRIEF DESCRIPTION OF THE DRAWINGS

FIG. 1A, FIG. 1B, and FIG. 1C shows illustrations of the transdermal delivery systems of the present invention.

FIG. 2 shows the XRPD of the donepezil hydrochloride polymorph Form III.

### DETAILED DESCRIPTION OF THE INVENTION

### I. General

The present invention provides a method of preparing the drug matrix layer using donepezil hydrochloride polymorph Form III.

### II. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

“High-energy surface treatment” refers to a process of increasing the surface energy of a surface through use of a high-energy treatment. A representative high-energy surface treatment includes a corona discharge treatment that involves exposing a surface to a corona discharge or corona plasma to modify the properties of the surface. Surfaces that are exposed to the high-energy surface treatment can be characterized by a higher surface energy, as measured by Dynes, compared to the surface energy prior to the high-energy surface treatment.

“Contact” refers to bringing two objects or surfaces of two objects into close proximity such that they are physically touching one another.

“Microporous membrane” refers to a membrane having a plurality of pores filled with a membrane solvent composition for transporting the active agent from the drug matrix layer to the contact adhesive layer and to the patient.

“Occlusive material” refers to a material that has a low moisture transmission rate to, for example, reduce or minimize moisture loss from skin. Occlusives can include materials such as silicones, waxes, oils, as well as a variety of polymers and copolymers.

“Surface energy” refers to the energy required to move an object across the surface. The surface energy is measured in Dynes, the force required to accelerate a mass of 1 gram at a rate of 1 centimeter per second squared (g·cm/s²). For example, 1 Dyne is equivalent to 1×10⁻⁵ Newtons.

“Alkaline salt” refers to a base such as sodium carbonate, sodium acetate, sodium bicarbonate, sodium hydroxide, sodium percarbonate, among others.

“D90 particle size” refers to the size distribution of a plurality of particles where 90% of the particles have a diameter of the stated D90 particle size or smaller.

“Line speed” refers to the speed at which the layer being exposed to the high-energy treatment is exposed to and removed from the high-energy treatment. Representative speeds can be inches or feet per minute.

“Laminating”, “laminate” or “lamination” refers to the process of preparing a material by combining two separate layers into one through use of heat, pressure or adhesives.

“Process liner” refers to a protective layer that is used before, during or after the laminating of two different layers to protect a surface of one of the layers. The process liner can then be removed from the surface prior to the next laminating step.

“Steady state flux” or “steady state equilibrium flux” refers to the flow of the active agent from the transdermal delivery system achieving a constant value without substantial changes over time.

“Unit dosage form” refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

An “adhesive matrix” as described herein includes matrices made in one piece, for example, matrices made via solvent casting or extrusion as well as matrices formed in two or more portions that are then pressed or joined together.

The term “therapeutically effective amount” as used herein refers to the amount of an active agent that is nontoxic but sufficient to provide the desired therapeutic effect. The amount that is “effective” will vary from subject to subject, depending on the age and general condition of the individual, the particular active agent or agents, and the like as known to those skilled in the art.

The phrase “pharmaceutically acceptable” is employed herein to refer to those compounds, salts, compositions, dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U. S. Pharmacopeia or other generally recognized pharmacopeia for use in mammals (e.g., animals), and more particularly, in humans.

The terms “transdermal” or “transdermal delivery” as used herein refer to administration of an active agent to a body surface of an individual so that the agent passes through the body surface, e.g., skin, and into the individual's blood stream. The term “transdermal” is intended to include transmucosal administration, i.e., administration of a drug to the mucosal (e.g., sublingual, buccal, vaginal, rectal) surface of an individual so that the agent passes through the mucosal tissue and into the individual's blood stream.

The terms “topical delivery system,” “transdermal delivery system” and “TDS,” which refer to the route of delivery of the drug via the skin tissue, are used interchangeably herein.

The terms “skin,” “tissue” or “cutaneous” tissue as used herein are defined as including tissues covered by a stratum comeum, or stratum lucidum, and/or other mucous membranes. The term further includes mucosal tissue, including the interior surface of body cavities, e.g., buccal, nasal, rectal, vaginal, etc., which have a mucosal lining. The term “skin” should be interpreted as including “mucosal tissue” and vice versa.

The terms “treat”, “treating”, “treatment,” “therapy,” “therapeutic” and the like, as used herein, encompass any course of medical intervention aimed at a pathologic condition, and includes not only permanent cure of a disease, but prevention of disease, control or even steps taken to mitigate a disease or disease symptoms. For instance, in reference to methods of treating a disorder, such as Alzheimer's disease, the embodiment, generally includes the administration of an active agent which reduces the frequency of, or delays the onset of, symptoms of the medical condition in a subject relative to a subject not receiving the active agent. This can include reversing, reducing, or arresting the symptoms, clinical signs, and underlying pathology of a condition in a manner to improve or stabilize a subject's condition (e.g., regression of mental facilities).

A “subject” or “patient” in whom administration of the therapeutic agent is an effective therapeutic regimen for a disease or disorder is preferably a human, but can be any animal, including a laboratory animal in the context of a trial or screening or activity experiment. Thus, as can be readily appreciated by one of ordinary skill in the art, the methods and systems as provided herein are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

“Therapeutic agent” refers to a drug or agent that can treat an injury, pathology, condition, or symptom (e.g., pain). Representative therapeutic agents include, but are not limited to, donepezil hydrochloride, donepezil free base, memantine, agents useful for treating Alzheimer's, and agents useful for treating other conditions and diseases.

“Molar ratio” refers to the ratio of the moles of a first component to the moles of a second component, where the molar ratio is determined by dividing the moles of the first component by the moles of the second component.

### III. Preparation of Drug Matrix Layer

The present invention provides a method of preparing a drug matrix layer including donepezil hydrochloride. Donepezil is an acetylcholinesterase inhibitor with the chemical structure 2,3-Dihydro-5,6-dimethoxy-2-[[1-(phenylmethyl)-4-piperidinyl]methyl]-IH-inden-1-one:

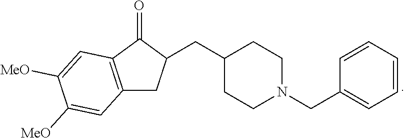 [SMILES: COc1cc2c(cc1OC)C(=O)C(CC1CCN(Cc3ccccc3)CC1)C2]

Donepezil has a molecular weight of 379.5 and is lipophilic (Log value 3.08-4.11). Donepezil hydrochloride has the following structure:

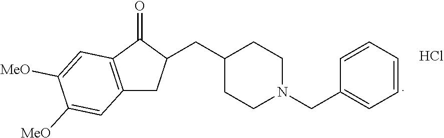 [SMILES: COc1cc2c(cc1OC)C(=O)C(CC1CCN(Cc3ccccc3)CC1)C2 ; Cl]

The donepezil hydrochloride can adopt one of several different crystalline polymorphic forms, or amorphous forms. U.S. Pat. Nos. 5,985,864 and 6,140,321, and PCT Publication No. WO 1997/046527, describe several crystalline polymorphic forms of donepezil hydrochloride, including Form I, Form II, Form III, Form IV, and Form V.

The drug matrix layer having donepezil hydrochloride can be prepared by a variety of methods. For example, donepezil hydrochloride polymorph Form I can be used as the starting point. Donepezil HCl polymorph Form I is characterized by an X-Ray Diffraction (XRD) Pattern having peaks at 9.9, 10.6, 12.7, 13.1, 13.7, 13.9, 14.9, 15.3, 16.1, 16.9, 17.5, 17.6, 18.4, 19.3, 19.8, 19.9, 21.2, 22.0, 22.5, 23.0, 23.6, 23.8, 23.9, 26.5, 28.0, and 29.5±0.2° 20. Alternatively, donepezil hydrochloride polymorph Form III can be used as the starting point.

In some embodiments, the present invention provides a method of preparing a drug matrix layer, comprising: forming a first mixture comprising donepezil HCl polymorph Form III, and sodium bicarbonate, thereby preparing the drug matrix layer.

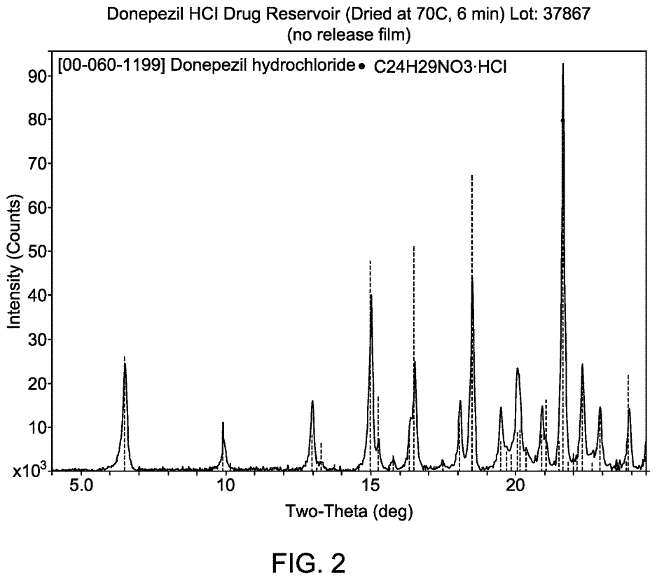

In some embodiments, the method of the present invention includes the method wherein the donepezil HCl polymorph Form III is characterized by an X-Ray Diffraction (XRD) Pattern having peaks at 6.6, 15.0, 16.5, 18.5, 20.1, 21.7, 26.0, and 28.2±0.2° 2θ. In some embodiments, the method of the present invention includes the method wherein the donepezil HCl polymorph Form III is further characterized by the XRD pattern having peaks at 18.1, 20.9, 22.3 and 27.2±0.2° 2θ. In some embodiments, the method of the present invention includes the method wherein the donepezil HCl polymorph Form III is characterized by the XRD pattern having peaks at 6.6, 15.0, 16.5, 18.1, 18.5, 20.1, 20.9, 21.7, 22.3, 26.0, 27.2 and 28.2±0.2° 2θ. In some embodiments, the method of the present invention includes the method wherein the donepezil HCl polymorph Form III is further characterized by the XRD pattern having peaks at 9.9, 13.0, 15.3, 15.7, 17.4, 19.5, 22.9, 23.9, 24.7 and 28.6±0.2° 2θ. In some embodiments, the method of the present invention includes the method wherein the donepezil HCl polymorph Form III is characterized by the XRD pattern having peaks at 6.6, 9.9, 13.0, 15.0, 15.3, 15.7, 16.5, 17.4, 18.1, 18.5, 19.5, 20.1, 20.9, 21.7, 22.3, 22.9, 23.9, 24.7, 26.0, 27.2, 28.2, and 28.6±0.2° 2θ. In some embodiments, the method of the present invention includes the method wherein the donepezil HCl polymorph Form III is characterized by the XRD pattern substantially as shown in FIG. 2.

The sodium carbonate can be present in the first reaction mixture in any suitable amount. For example, the sodium bicarbonate can be present in a molar ratio of from 2.0 to 0.5 relative to the donepezil HCl, or a molar ratio of from 1.9 to 0.6, 1.8 to 0.7, 1.7 to 0.8, 1.6 to 0.8, 1.5 to 0.8, 1.4 to 0.8, 1.3 to 0.8, 1.2 to 0.8, 1.1 to 0.8, 1.6 to 0.9, 1.5 to 1.0, 1.4 to 1.0, 1.3 to 1.0, 1.2 to 1.0, or from 1.1 to 1.0. The sodium bicarbonate can be present in a molar ratio of from 2.0 to 0.9 relative to the donepezil HCl, or a molar ratio of from 1.9 to 0.9, 1.8 to 0.9, 1.7 to 0.9, 1.6 to 0.9, 1.5 to 0.9, 1.4 to 0.9, 1.3 to 0.9, 1.2 to 0.9, or from 1.1 to 0.9. relative to the donepezil HCl. The sodium bicarbonate can be present in a molar ratio of from 1.1 to 0.7 relative to the donepezil HCl, or a molar ratio of from 1.1 to 0.75, 1.0 to 0.80, 1.0 to 0.85, or from 1.0 to 0.90 relative to the donepezil HCl. The sodium bicarbonate can also be present in a molar ratio of about 1.5, or 1.45, 1.4, 1.35, 1.3, 1.25, 1.2, 1.15, 1.1, 1.05, 1.0. 0.95, or about 0.9 relative to the donepezil HCl. The sodium bicarbonate can also be present in a molar ratio of about 1.1, or 1.05, 1.0, 0.95, 0.94, 0.93, 0.92, 0.91, 0.90, 0.89, 0.88, 0.87, 0.86, 0.85, 0.84, 0.83, 0.82, 0.81, or about 0.8 relative to the donepezil HCl.

In some embodiments, the method of the present invention includes the method wherein the sodium bicarbonate can be present in the first reaction mixture in a molar ratio of from 1.5 to 0.9 to the donepezil HCl. In some embodiments, the method of the present invention includes the method wherein the sodium bicarbonate can be present in the first reaction mixture in a molar ratio of from 1.4 to 0.9 to the donepezil HCl. In some embodiments, the method of the present invention includes the method wherein the sodium bicarbonate can be present in the first reaction mixture in a molar ratio of from 1.3 to 0.9 to the donepezil HCl. In some embodiments, the method of the present invention includes the method wherein the sodium bicarbonate can be present in the first reaction mixture in a molar ratio of from 1.2 to 0.9 to the donepezil HCl. In some embodiments, the method of the present invention includes the method wherein the sodium bicarbonate can be present in the first reaction mixture in a molar ratio of from 1.1 to 0.9 to the donepezil HCl. In some embodiments, the method of the present invention includes the method wherein the sodium bicarbonate can be present in the first reaction mixture in a molar ratio of from 1.05 to 0.95 to the donepezil HCl. In some embodiments, the method of the present invention includes the method wherein the sodium bicarbonate can be present in the first reaction mixture in a molar ratio of from 1.1 to 1.0 to the donepezil HCl.

In some embodiments, the method of the present invention includes the method wherein the sodium bicarbonate can be present in the first reaction mixture in a molar ratio of at least 0.85 to the donepezil HCl. In some embodiments, the method of the present invention includes the method wherein the sodium bicarbonate can be present in the first reaction mixture in a molar ratio of about 0.88 to the donepezil HCl. In some embodiments, the method of the present invention includes the method wherein the sodium bicarbonate can be present in the first reaction mixture in a molar ratio of 0.88 to the donepezil HCl. In some embodiments, the method of the present invention includes the method wherein the sodium bicarbonate can be present in the first reaction mixture in a molar ratio of about 0.92 to the donepezil HCl. In some embodiments, the method of the present invention includes the method wherein the sodium bicarbonate can be present in the first reaction mixture in a molar ratio of 0.92 to the donepezil HCl. In some embodiments, the method of the present invention includes the method wherein the sodium bicarbonate can be present in the first reaction mixture in a molar ratio of about 0.95 to the donepezil HCl. In some embodiments, the method of the present invention includes the method wherein the sodium bicarbonate can be present in the first reaction mixture in a molar ratio of 0.95 to the donepezil HCl.

In some embodiments, the method of the present invention includes the method wherein the sodium bicarbonate can be present in the first reaction mixture in a molar ratio of at least 1.0 to the donepezil HCl. In some embodiments, the method of the present invention includes the method wherein the sodium bicarbonate can be present in the first reaction mixture in a molar ratio of about 1.0 to the donepezil HCl. In some embodiments, the method of the present invention includes the method wherein the sodium bicarbonate can be present in the first reaction mixture in a molar ratio of 1.0 to the donepezil HCl.

In some embodiments, the method of the present invention includes the method wherein the sodium bicarbonate can be present in the first reaction mixture in a molar ratio of at least 0.85 to the donepezil HCl. In some embodiments, the method of the present invention includes the method wherein the sodium bicarbonate can be present in the first reaction mixture in a molar ratio of about 0.88 to the donepezil HCl. In some embodiments, the method of the present invention includes the method wherein the sodium bicarbonate can be present in the first reaction mixture in a molar ratio of 0.88 to the donepezil HCl. In some embodiments, the method of the present invention includes the method wherein the sodium bicarbonate can be present in the first reaction mixture in a molar ratio of about 0.92 to the donepezil HCl. In some embodiments, the method of the present invention includes the method wherein the sodium bicarbonate can be present in the first reaction mixture in a molar ratio of 0.92 to the donepezil HCl. In some embodiments, the method of the present invention includes the method wherein the sodium bicarbonate can be present in the first reaction mixture in a molar ratio of 0.95 to the donepezil HCl.

In some embodiments, the method of the present invention includes the method comprising forming the first mixture comprising donepezil HCl polymorph Form III, and sodium bicarbonate, wherein the sodium bicarbonate is present in a molar ratio of 1.0 to the donepezil HCl. In some embodiments, the method of the present invention includes the method comprising forming the first mixture comprising donepezil HCl polymorph Form III, and sodium bicarbonate, wherein the sodium bicarbonate is present in a molar ratio of 0.88 to the donepezil HCl. In some embodiments, the method of the present invention includes the method comprising forming the first mixture comprising donepezil HCl polymorph Form III, and sodium bicarbonate, wherein the sodium bicarbonate is present in a molar ratio of 0.92 to the donepezil HCl. In some embodiments, the method of the present invention includes the method comprising forming the first mixture comprising donepezil HCl polymorph Form III, and sodium bicarbonate, wherein the sodium bicarbonate is present in a molar ratio of 0.95 to the donepezil HCl.

The drug matrix layer prepared by the methods of the present invention can include other components, such as, but not limited to, an adhesive matrix, an acrylate polymer, a drug matrix solvent composition, an alkaline salt, and others as described within. In some embodiments, the method of the present invention includes the method further comprising prior to adding the sodium bicarbonate to the first reaction mixture: adding one or more of triethyl citrate, lauryl lactate, ascorbyl palmitate, polyvinylpyrrolidone, and sorbitan monolaurate to the first mixture.

In some embodiments, the method of the present invention includes the method further comprising prior to adding the sodium bicarbonate to the first reaction mixture: adding triethyl citrate, lauryl lactate and ethyl acetate to the first mixture. In some embodiments, the method of the present invention includes the method further comprising prior to adding the sodium bicarbonate to the first reaction mixture: adding ascorbyl palmitate to the first mixture. In some embodiments, the method of the present invention includes the method further comprising prior to adding the sodium bicarbonate to the first reaction mixture: adding polyvinylpyrrolidone to the first mixture. In some embodiments, the method of the present invention includes the method further comprising prior to adding the sodium bicarbonate to the first reaction mixture: adding sorbitan monolaurate to the first mixture. In some embodiments, the method of the present invention includes the method further comprising adding an acrylate polymer to the first mixture.

In some embodiments, the method of the present invention includes the method further comprising
adding triethyl citrate, lauryl lactate and ethyl acetate to the first mixture;
adding ascorbyl palmitate to the first mixture;
adding polyvinylpyrrolidone to the first mixture;
adding sorbitan monolaurate to the first mixture; and
adding an acrylate polymer to the first mixture, thereby preparing the drug matrix layer.

In some embodiments, the method of the present invention includes the method comprising
forming a first mixture comprising triethyl citrate, lauryl lactate and ethyl acetate;
adding ascorbyl palmitate to the first mixture;
adding polyvinylpyrrolidone to the first mixture;
adding donepezil HCl polymorph Form III to the first mixture;
adding sorbitan monolaurate to the first mixture;
adding sodium bicarbonate and glycerin to the first mixture, wherein the sodium bicarbonate is present in a molar ratio of about 1.0 to the donepezil HCl; and
adding an acrylate polymer to the first mixture, thereby preparing the drug matrix layer.

In some embodiments, the method of the present invention includes the method comprising
forming a first mixture comprising triethyl citrate, lauryl lactate and ethyl acetate;

adding ascorbyl palmitate to the first mixture;

adding polyvinylpyrrolidone to the first mixture;

adding donepezil HCl polymorph Form III to the first mixture;

adding sorbitan monolaurate to the first mixture;

adding sodium bicarbonate and glycerin to the first mixture, wherein the sodium bicarbonate is present in a molar ratio of about 0.88 to the donepezil HCl; and
adding an acrylate polymer to the first mixture, thereby preparing the drug matrix layer.

In some embodiments, the method of the present invention includes the method comprising
forming a first mixture comprising triethyl citrate, lauryl lactate and ethyl acetate;
adding ascorbyl palmitate to the first mixture;
adding polyvinylpyrrolidone to the first mixture;
adding donepezil HCl polymorph Form III to the first mixture;
adding sorbitan monolaurate to the first mixture;
adding sodium bicarbonate and glycerin to the first mixture, wherein the sodium bicarbonate is present in a molar ratio of about 0.92 to the donepezil HCl; and
adding an acrylate polymer to the first mixture, thereby preparing the drug matrix layer.

In some embodiments, the method of the present invention includes the method comprising
forming a first mixture comprising triethyl citrate, lauryl lactate and ethyl acetate;
adding ascorbyl palmitate to the first mixture;
adding polyvinylpyrrolidone to the first mixture;
adding donepezil HCl polymorph Form III to the first mixture;
adding sorbitan monolaurate to the first mixture;
adding sodium bicarbonate and glycerin to the first mixture, wherein the sodium bicarbonate is present in a molar ratio of about 0.95 to the donepezil HCl; and
adding an acrylate polymer to the first mixture, thereby preparing the drug matrix layer.

In some embodiments, the method of the present invention includes the method further comprising coating the first mixture on a release liner; and drying the coated mixture.

### IV. Preparation of Transdermal Delivery System

The transdermal delivery system of the present invention can be prepared by any suitable means known to one of skill in the art.

The thickness and/or size of the device and/or adhesive matrices may be determined by one skilled in the art based at least on considerations of wearability and/or required dose. It will be appreciated that the administration site for the device will affect the wearability considerations due to the available size of the administration site and the use of the administration site (e.g. need for flexibility to support movement). In some embodiments, the device and/or adhesive matrix has a thickness of between about 25-500 μm. In some embodiments, the device and/or adhesive matrix has a thickness of between about 50-500 μm. In some embodiments, the patch has a size in the range of about 16 cm²-225 cm². It will be appreciated that the thickness and size provided here are merely exemplary and the actual thickness and or size may be thinner/smaller or thicker/larger as needed for a specific formulation.

Fabrication of a transdermal delivery system is routinely done by skilled artisans and involves casting or extruding each of the adhesive layers onto a suitable film such as a release liner or onto another layer of the transdermal delivery system, and drying if needed to remove solvents and/or volatile compounds. Layers of the transdermal delivery system can be laminated together to form the final system.

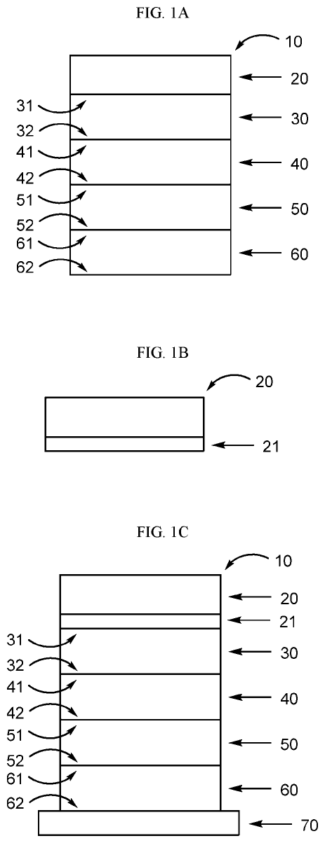

Transdermal delivery systems and drug adhesive matrices were prepared to illustrate the embodiments described herein. The Examples set forth exemplary compositions and delivery systems. As described in Example 1, a transdermal delivery system comprised a drug matrix layer and a contact adhesive layer with a rate controlling membrane situated between the drug matrix layer and the contact adhesive layer, as depicted in FIG. 1A. A drug matrix layer in the form of a solid monolithic adhesive reservoir was prepared using an acrylate/vinyl acetate copolymer adhesive with drug matrix solvent composition-triethyl citrate, lauryl lactate and ethyl acetate. A contact adhesive layer comprised of the same acrylate/vinyl acetate copolymer adhesive, along with triethyl citrate, lauryl lactate and ethyl acetate as drug matrix solvent composition was prepared. A rate controlling membrane, to control the diffusional release of donepezil free base from the drug matrix layer, separated the drug matrix layer and the contact adhesive layer.

The transdermal delivery system can be prepared by any suitable means. In some embodiments, the present invention includes a method for preparing a transdermal delivery system, comprising:
(i) laminating a microporous membrane layer onto a top surface of a contact adhesive layer to form a contact adhesive laminate having a top surface and a bottom surface;
(ii) laminating a drug matrix layer onto the top surface of the contact adhesive laminate to form a drug matrix laminate having a top surface and a bottom surface;
(iii) laminating a separating layer onto the top surface of the drug matrix laminate to form an active laminate having a top surface and a bottom surface, wherein the separating layer comprises a top surface and a bottom surface, wherein the top surface of the separating layer comprises a coating of ethylene-vinyl acetate copolymer, and wherein the bottom surface of the separating layer is in contact with the top surface of the drug matrix laminate;
(iv) laminating a polyester fabric onto an adhesive overlay layer comprising acrylate polymer to form a backing layer having a top surface and a bottom surface;
(v) laminating the bottom surface of the backing layer onto the top surface of the active laminate so that the adhesive overlay layer is in contact with the top surface of the active laminate, thereby forming the transdermal delivery system of the present invention.

The method can include additional steps, such as treating the separating layer with a high-energy surface treatment. In some embodiments, the method further comprises before laminating the separating layer onto the top surface of the drug matrix layer: (vi) treating the top surface of the separating layer with a high-energy surface treatment to form a treated separating layer, wherein the treated separating layer comprises a top surface and a bottom surface.

In some embodiments, the present invention includes a method for preparing a transdermal delivery system, comprising:
(i) laminating a microporous membrane layer onto a top surface of a contact adhesive layer to form a contact adhesive laminate having a top surface and a bottom surface;
(ii) laminating a drug matrix layer onto the top surface of the contact adhesive laminate to form a drug matrix laminate having a top surface and a bottom surface;
(iii) treating a top surface of a separating layer with a high-energy surface treatment to form a treated separating layer, wherein the top surface of the separating layer comprises a coating of ethylene-vinyl acetate copolymer, and wherein the treated separating layer comprises a top surface and a bottom surface; and
(iv) laminating the treated separating layer onto the top surface of the drug matrix laminate to form an active laminate having a top surface and a bottom surface, wherein the bottom surface of the treated separating layer is in contact with the top surface of the drug matrix laminate;
(v) laminating a polyester fabric onto an adhesive overlay layer comprising acrylate polymer to form a backing layer having a top surface and a bottom surface;
(vi) laminating the bottom surface of the backing layer onto the top surface of the treated active laminate so that the adhesive overlay layer is in contact with the top surface of the treated active laminate, thereby forming the transdermal delivery system of the present invention.

The top-surface of the separating layer can be treated with any suitable high-energy surface treatment to form the treated separating layer. In some embodiments, the high-energy surface treatment is selected from the group consisting of corona discharge treatment, plasma treatment, UV radiation, ion beam treatment, electron beam treatment and combinations thereof. In some embodiments, the high-energy surface treatment is corona discharge treatment.

The corona discharge treatment can be performed using a variety of process parameters, including power, line speed, and width of the corona treatment electrodes, to achieve any suitable power density. Representative power densities include, but are not limited to, from 0.1 to 10 W/ft²/min, or from 0.5 to 10, or from 0.6 to 9, or from 0.7 to 8, or from 0.8 to 7, or from 0.9 to 6, or from 1 to 5, or from 1.55 to 4, or from 2 to 3, or from 2.1 to 2.9, or from 2.1 to 2.8, or from 2.1 to 2.7, or from 2.1 to 2.6 W/ft²/min. Other power densities include, but are not limited to, about 1 W/ft²/min, or about 1.5, 1.6, 1.7, 1.8, 1.9, 2.0, 2.1, 2.2, 2.3, 2.4, 2.5, 2.6, 2.7, 2.8, 2.9, or about 3.0 W/ft²/min.

The corona discharge treatment can be performed under any suitable conditions. Representative conditions include, but are not limited to, power and line speed. Representative power includes, but is not limited to, 0.001 kW to 1.0 kW, or 0.01 to 1.0, or 0.01 to 0.9, 0.01 to 0.8, 0.01 to 0.7, 0.01 to 0.6, 0.01 to 0.5, 0.02 to 0.04, 0.03 to 0.3, 0.04 to 0.25, 0.05 to 0.20, 0.06 to 0.15, 0.07 to 0.14, 0.08 to 0.13, 0.09 to 0.12, or 0.1 to 1.2 kW. In some embodiments, the corona discharge treatment is performed using a power of from 0.01 kW to 1.0 kW. In some embodiments, the corona discharge treatment is performed using a power of from 0.05 kW to 0.12 kW. In some embodiments, the corona discharge treatment is performed using a power of from 0.10 KW to 0.12 kW. In some embodiments, the corona discharge treatment is performed using a power of about 0.11 kW. In some embodiments, the corona discharge treatment is performed using a power of about 0.24 kW.

Representative line speed for the corona discharge treatment includes, but is not limited to, 1 to 100 feet per minute, or 1 to 95, 1 to 90, 1 to 85, 1 to 80, 1 to 75, 1 to 70, 1 to 65, 1 to 60, 1 to 55, 5 to 50, 5 to 45, 5 to 40, 5 to 35, 5 to 30, 5 to 25, 5 to 20, 6 to 19, 7 to 18, 8 to 17, 9 to 16, 10 to 15, or 11 to 14 feet per minute. Other representative line speeds include, but are not limited to, 10 to 50 feet per minute, or 15 to 45, or 20 to 40 feet per minute. Other representative line speeds include, but are not limited to, 10 feet per minutes, or 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 feet per minutes.

In some embodiments, the corona discharge treatment is performed using a line speed of 1 to 100 feet per minute. In some embodiments, the corona discharge treatment is performed using a line speed of 20 to 40 feet per minute. In some embodiments, the corona discharge treatment is performed using a line speed of about 30 feet per minute. In some embodiments, the corona discharge treatment is performed using a line speed of about 13 feet per minute.

The corona discharge treatment provides a treated separating layer having any suitable surface energy. A representative surface energy of the treated separating layer includes, but is not limited to, at least 10 Dynes, or at least 15, 20, 25, 30, 31, 32, 33, 34, 35, 36, 37, 38, 39, 40, 41, 42, 43, 44, 45, 46, 47, 48, 49, 50, 55, 60, 65, 70, or at least 75 Dynes. In some embodiments, the top surface of the treated separating layer has a surface energy of at least 30 Dynes. In some embodiments, the top surface of the treated separating layer has a surface energy of at least 35 Dynes. In some embodiments, the top surface of the treated separating layer has a surface energy of at least 40 Dynes.

In some embodiments, the top surface of the treated separating layer has a surface energy that is greater than the top surface of the separating layer prior to the high-energy surface treatment. The top surface of the treated separating layer can have a surface energy at least 1 Dyne greater than the top surface of the separating layer prior to the high-energy surface treatment, or at least 2, 3, 4, 5, 6, 7, 8, 9, 10, 11, 12, 13, 14, 15, 16, 17, 18, 19, 20, 25, or 30 Dynes greater than the top surface of the separating layer prior to the high-energy surface treatment. In some embodiments, the top surface of the treated separating layer has a surface energy at least 5 Dynes greater than the top surface of the separating layer prior to the high-energy surface treatment. In some embodiments, the top surface of the treated separating layer has a surface energy at least 10 Dynes greater than the top surface of the separating layer prior to the high-energy surface treatment. In some embodiments, the top surface of the treated separating layer has a surface energy at least 15 Dynes greater than the top surface of the separating layer prior to the high-energy surface treatment. In some embodiments, the top surface of the treated separating layer has a surface energy at least 20 Dynes greater than the top surface of the separating layer prior to the high-energy surface treatment.

In some embodiments, the bottom surface of the contact adhesive layer is in contact with a first process liner.

In some embodiments, the method of preparing the transdermal delivery system includes: (vii) removing the first process liner to expose the bottom surface of the contact adhesive layer; and (viii) laminating a release liner onto the bottom surface of the contact adhesive layer.

In some embodiments, the present invention provides a transdermal delivery system of the present invention prepared by the methods of the present invention.

In some embodiments, the present invention provides a method for preparing a transdermal delivery system, comprising:
(i) laminating a microporous membrane layer onto a top surface of a contact adhesive layer to form a contact adhesive laminate having a top surface and a bottom surface;
(ii) preparing a drug matrix layer comprising:
   forming a first mixture comprising triethyl citrate, lauryl lactate, and ethyl acetate,
   adding ascorbyl palmitate to the first mixture,
   adding polyvinylpyrrolidone to the first mixture,
   adding donepezil HCl polymorph Form III to the first mixture,
   adding sorbitan monolaurate to the first mixture,
   adding sodium bicarbonate and glycerin to the first mixture, wherein the
   sodium bicarbonate is present in a molar ratio of at least 1.0 to the donepezil HCl,
   adding an acrylate polymer to the first mixture,
   coating the first mixture on a release liner,
   drying the coated mixture, and
   removing the release liner, thereby preparing the drug matrix layer;
(iii) laminating the drug matrix layer onto the top surface of the contact adhesive laminate to form a drug matrix laminate having a top surface and a bottom surface;
(iv) laminating a separating layer onto the top surface of the drug matrix laminate to form an active laminate having a top surface and a bottom surface, wherein the separating layer comprises a top surface and a bottom surface, wherein the top surface of the separating layer comprises a coating of ethylene-vinyl acetate copolymer, and wherein the bottom surface of the separating layer is in contact with the top surface of the drug matrix laminate;
(v) laminating a polyester fabric onto an adhesive overlay layer comprising acrylate polymer to form a backing layer having a top surface and a bottom surface;
(vi) laminating the bottom surface of the backing layer onto the top surface of the active laminate so that the adhesive overlay layer is in contact with the top surface of the active laminate;
(vii) treating the top surface of the separating layer with a corona discharge treatment to form a treated separating layer,
   wherein the corona discharge treatment is performed using a power of from 0.10 kW to 0.12 kW and a power density of from 2.1 to 2.6 W/ft²/min,
   wherein the treated separating layer comprises a top surface and a bottom surface such that the top surface of the treated separating layer has a surface energy of at least 40 Dynes, and
   wherein the bottom surface of the contact adhesive layer is in contact with a first process liner;
(viii) removing the first process liner to expose the bottom surface of the contact adhesive layer; and
(ix) laminating a release liner onto the bottom surface of the contact adhesive layer, thereby forming the transdermal delivery system.

In some embodiments, the present invention provides a method for preparing a transdermal delivery system, comprising:
(i) laminating a microporous membrane layer onto a top surface of a contact adhesive layer to form a contact adhesive laminate having a top surface and a bottom surface;
(ii) preparing a drug matrix layer comprising:
   forming a first mixture comprising triethyl citrate, lauryl lactate, and ethyl acetate,
   adding ascorbyl palmitate to the first mixture,
   adding polyvinylpyrrolidone to the first mixture,
   adding donepezil HCl polymorph Form III to the first mixture,
   adding sorbitan monolaurate to the first mixture,
   adding sodium bicarbonate and glycerin to the first mixture, wherein the
   sodium bicarbonate is present in a molar ratio of at least 0.85 to the donepezil HCl,
   adding an acrylate polymer to the first mixture,
   coating the first mixture on a release liner,
   drying the coated mixture, and
   removing the release liner, thereby preparing the drug matrix layer;
(iii) laminating the drug matrix layer onto the top surface of the contact adhesive laminate to form a drug matrix laminate having a top surface and a bottom surface;
(iv) laminating a separating layer onto the top surface of the drug matrix laminate to form an active laminate having a top surface and a bottom surface, wherein the separating layer comprises a top surface and a bottom surface, wherein the top surface of the separating layer comprises a coating of ethylene-vinyl acetate copolymer, and wherein the bottom surface of the separating layer is in contact with the top surface of the drug matrix laminate;
(v) laminating a polyester fabric onto an adhesive overlay layer comprising acrylate polymer to form a backing layer having a top surface and a bottom surface;
(vi) laminating the bottom surface of the backing layer onto the top surface of the active laminate so that the adhesive overlay layer is in contact with the top surface of the active laminate;
(vii) treating the top surface of the separating layer with a corona discharge treatment to form a treated separating layer,
   wherein the corona discharge treatment is performed using a power of from 0.10 KW to 0.12 kW and a power density of from 2.1 to 2.6 W/ft²/min,
   wherein the treated separating layer comprises a top surface and a bottom surface such that the top surface of the treated separating layer has a surface energy of at least 40 Dynes, and
   wherein the bottom surface of the contact adhesive layer is in contact with a first process liner;
(viii) removing the first process liner to expose the bottom surface of the contact adhesive layer; and
(ix) laminating a release liner onto the bottom surface of the contact adhesive layer, thereby forming the transdermal delivery system.

In some embodiments, the present invention provides a method for preparing a transdermal delivery system, comprising:
(i) laminating a microporous membrane layer onto a top surface of a contact adhesive layer to form a contact adhesive laminate having a top surface and a bottom surface;
(ii) preparing a drug matrix layer comprising:
   forming a first mixture comprising triethyl citrate, lauryl lactate, and ethyl acetate,
   adding ascorbyl palmitate to the first mixture,
   adding polyvinylpyrrolidone to the first mixture,
   adding donepezil HCl polymorph Form III to the first mixture,
   adding sorbitan monolaurate to the first mixture,
   adding sodium bicarbonate and glycerin to the first mixture, wherein the sodium bicarbonate is present in a molar ratio of at least 0.90 to the donepezil HCl,
   adding an acrylate polymer to the first mixture,
   coating the first mixture on a release liner,
   drying the coated mixture, and
   removing the release liner, thereby preparing the drug matrix layer;
(iii) laminating the drug matrix layer onto the top surface of the contact adhesive laminate to form a drug matrix laminate having a top surface and a bottom surface;
(iv) laminating a separating layer onto the top surface of the drug matrix laminate to form an active laminate having a top surface and a bottom surface, wherein the separating layer comprises a top surface and a bottom surface, wherein the top surface of the separating layer comprises a coating of ethylene-vinyl acetate copolymer, and wherein the bottom surface of the separating layer is in contact with the top surface of the drug matrix laminate;
(v) laminating a polyester fabric onto an adhesive overlay layer comprising acrylate polymer to form a backing layer having a top surface and a bottom surface;
(vi) laminating the bottom surface of the backing layer onto the top surface of the active laminate so that the adhesive overlay layer is in contact with the top surface of the active laminate;
(vii) treating the top surface of the separating layer with a corona discharge treatment to form a treated separating layer,
   wherein the corona discharge treatment is performed using a power of from 0.10 KW to 0.12 KW and a power density of from 2.1 to 2.6 W/ft²/min,
   wherein the treated separating layer comprises a top surface and a bottom surface such that the top surface of the treated separating layer has a surface energy of at least 40 Dynes, and
   wherein the bottom surface of the contact adhesive layer is in contact with a first process liner;
(viii) removing the first process liner to expose the bottom surface of the contact adhesive layer; and
(ix) laminating a release liner onto the bottom surface of the contact adhesive layer, thereby forming the transdermal delivery system.

In another embodiment, the present invention provides a transdermal delivery system prepared by the method of the present invention.

### V. Transdermal Delivery System

A transdermal delivery system for systemic delivery of water-insoluble drug base is provided. The transdermal system in general is comprised of a contact adhesive layer and a drug matrix layer, where the two layers are separated by a membrane layer that includes a microporous membrane that has been pretreated with a membrane solvent composition. The system can include additional layers as are described below. The composition of the layers in the system are now described.

The transdermal delivery system of the present invention can have a variety of configurations, as shown in FIG. 1A-FIG. 1C. FIG. 1A shows a transdermal delivery system 10 having a backing layer 20, a separating layer 30 having a top surface 31 and a bottom surface 32, a drug matrix layer 40 having a top surface 41 and a bottom surface 42, a membrane layer 50 having a top surface 51 and a bottom surface 52, and a contact adhesive layer 60 having a top surface 61 and a bottom surface 62.

In some embodiments, the present invention provides a transdermal delivery system, comprising:
(1) a backing layer;
(2) a separating layer treated with a high-energy surface treatment, wherein the separating layer has a top surface and a bottom surface such that the top surface is in contact with the backing layer;
(3) a drug matrix layer comprising donepezil HCl, wherein the adhesive contact layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer.

In some embodiments, the present invention provides a transdermal delivery system, comprising:
(1) a backing layer;
(2) a separating layer, wherein the separating layer has a top surface and a bottom surface such that the top surface is in contact with the backing layer;
(3) a drug matrix layer comprising donepezil HCl, and donepezil free base wherein the drug matrix layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer, wherein the contact adhesive layer comprises donepezil free base in an amount of at least 0.1% (w/w) of the total weight of the contact adhesive layer.

In some embodiments, the present invention provides a transdermal delivery system, comprising:
(1) a backing layer;
(2) a separating layer, wherein the separating layer has a top surface and a bottom surface such that the top surface is in contact with the backing layer;
(3) a drug matrix layer comprising donepezil HCl, and donepezil free base wherein the drug matrix layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer,
   wherein the contact adhesive layer comprises donepezil free base in an amount of from 0.1 to 10% (w/w) of the total weight of the contact adhesive layer.

The transdermal delivery system of the present invention can have a variety of configurations, as shown in FIG. 1A-FIG. 1C. FIG. 1A shows a transdermal delivery system 10 having a backing layer 20, a separating layer 30 having a top surface 31 and a bottom surface 32, a drug matrix layer 40 having a top surface 41 and a bottom surface 42, a membrane layer 50 having a top surface 51 and a bottom surface 52, and a contact adhesive layer 60 having a top surface 61 and a bottom surface 62.

### Backing Layer

The transdermal delivery system can comprise a backing layer that provides a structural element for holding or supporting the underlying adhesive layer(s). The backing layer may be formed of any suitable material as known in the art. In some embodiments, the backing layer is occlusive. In some embodiments, the backing is preferably impermeable or substantially impermeable to moisture. In one exemplary embodiment, the backing layer has a moisture vapor transmission rate of less than about 50 g/m2-day. In some embodiments, the backing layer is inert. In some embodiments, the backing layer preferably prevents release of components of the adhesive layer through the backing layer. The backing layer may be flexible or nonflexible. The backing layer is preferably at least partially flexible such that the backing layer is able to conform at least partially to the shape of the skin where the patch is applied. In some embodiments, the backing layer is flexible such that the backing layer conforms to the shape of the skin where the patch is applied. In some embodiments, the backing layer is sufficiently flexible to maintain contact at the application site with movement, e.g. skin movement. Typically, the material used for the backing layer should permit the device to follow the contours of the skin or other application site and be worn comfortably on areas of skin such as at joints or other points of flexure, that are normally subjected to mechanical strain with little or no likelihood of the device disengaging from the skin due to differences in the flexibility or resiliency of the skin and the device.

In some embodiments, the backing layer comprises an elastic polymer film, a polymer fabric, a multi-directional elastic woven fabric, a multi-directional elastic nonwoven fabric, a stretchable polymer film, a stretchable woven fabric, or a stretchable nonwoven fabric

In some embodiments, the backing layer is formed of one or more of a film, non-woven fabric, woven fabric, laminate, and combinations thereof. In some embodiments, the film is a polymer film comprised of one or more polymers. Suitable polymers are known in the art and include elastomers, polyesters, polyethylene, polypropylene, polyurethanes and polyether amides. In some embodiments, the backing layer is formed of one or more of polyethylene terephthalate, various nylons, polypropylene, metalized polyester films, polyvinylidene chloride, and aluminum foil. In some embodiments, the backing layer is a fabric formed of one or more of polyesters such as polyethylene terephthalate, polyurethane, polyvinyl acetate, polyvinylidene chloride and polyethylene. In some embodiments, the backing layer comprises one or more polymers of polyesters, polyethylenes, polypropylenes, polyvinylchloride, polyethylene vinyl acetate or copolymers thereof, or polyurethanes. In some embodiments, the backing layer is formed of a polyester film laminate. In some embodiments, the backing layer is formed of a laminate of polyester and ethylene vinyl acetate copolymer (EVA) heat seal layers (9% EVA). One particular polyester film laminate is the polyethylene and polyester laminate such as the laminate sold under the name SCOTCHPAK™ #9723. In some embodiments, the backing layer includes KOB 052. In some embodiments, the backing layer includes SCOTCHPAK™ #9732.

In some embodiments, the backing layer has a thickness of about 0.2-50 millimeters.

The transdermal delivery system can include an adhesive overlay. In some embodiments, the backing layer further comprises an adhesive overlay layer in contact with the top surface of the separating layer.

The backing layer can adopt a variety of configurations, such as shown in FIG. 1B. FIG. 1B shows the backing layer 20 having an adhesive overlay layer 21.

The adhesive component in the backing layer can be any of a variety of adhesive materials, such as pressure sensitive adhesive polymers. Polyacrylate pressure sensitive adhesive polymers are an example, and typically comprise a polyacrylate that is a polymer or a copolymer of a monomer or monomers selected from acrylic acid esters and methacrylic acid esters. Other monomers, such as acrylic acid and vinyl acetate, may be present. In some embodiments, the acrylic polymer is based on acrylic esters such as 2-ethylhexyl acrylate (2-EHA) and ethyl acrylate. In some embodiments, the polyacrylate polymer is a polymer or a copolymer of a monomer or monomers selected from acrylic acid and vinyl acetate. In some embodiments, the acrylic polymer adhesive has pendent carboxyl (—COOH) or hydroxyl (—OH) functional groups. In some embodiments, the acrylic polymer adhesive comprises at least one of polyacrylate, polymethacrylate, derivatives thereof, and co-polymers thereof. In some embodiments, the acrylic adhesive is comprised of an acrylate copolymer comprising acrylic ester monomers, acrylic acid, and/or vinyl acetate monomers. A copolymer of acrylic acid and vinyl acetate is one example. Acrylate copolymers are sold under the trade-name DURO-TAK® and include, but are not limited to, DURO-TAK 87-2287, 387-2516, 387-2051, and 387-2074. In some embodiments, the acrylate polymer comprises DURO-TAK 82-2287. In some embodiments, the acrylate polymer comprises DURO-TAK 87-2052/2287/2051.

In some embodiments, the adhesive overlay layer comprises an acrylate copolymer.

### Separating Layer

In some embodiments, the top surface of the separating layer is treated with a high-energy surface treatment. In some embodiments, the transdermal delivery system includes a separating layer treated with a high-energy surface treatment, wherein the separating layer has a top surface and a bottom surface such that the top surface is in contact with the backing layer. The separating layer may be formed of any suitable material as known in the art. In some embodiments, the separating layer comprises at least one of an occlusive material or a breathable material.

In some embodiments, the separating layer is occlusive. In some embodiments, the backing is preferably impermeable or substantially impermeable to moisture. In one exemplary embodiment, the backing layer has a moisture vapor transmission rate of less than about 50 g/cm2-day. In some embodiments, the separating layer is preferably inert and/or does not absorb components of the adhesive layer, including the active agent. In some embodiments, the separating layer preferably prevents release of components of the adhesive layer through the separating layer. The separating layer may be flexible or nonflexible. The separating layer is preferably at least partially flexible such that the separating layer is able to conform at least partially to the shape of the skin where the patch is applied. In some embodiments, the separating layer is flexible such that the separating layer conforms to the shape of the skin where the patch is applied. In some embodiments, the separating layer is sufficiently flexible to maintain contact at the application site with movement, e.g. skin movement. Typically, the material used for the separating layer should permit the device to follow the contours of the skin or other application site and be worn comfortably on areas of skin such as at joints or other points of flexure, that are normally subjected to mechanical strain with little or no likelihood of the device disengaging from the skin due to differences in the flexibility or resiliency of the skin and the device.

In some embodiments, the separating layer comprises an elastic polymer film, a polymer fabric, a multi-directional elastic woven fabric, a multi-directional elastic nonwoven fabric, a stretchable polymer film, a stretchable woven fabric, or a stretchable nonwoven fabric. In some embodiments, the separating layer is formed of one or more of a film, non-woven fabric, woven fabric, laminate, and combinations thereof. In some embodiments, the film is a polymer film comprised of one or more polymers. Suitable polymers are known in the art and include elastomers, polyesters, polyethylene, polypropylene, polyurethanes and polyether amides. In some embodiments, the separating layer is formed of one or more of polyethylene terephthalate, various nylons, polypropylene, metalized polyester films, polyvinylidene chloride, and aluminum foil. In some embodiments, the separating layer is a fabric formed of one or more of polyesters such as polyethylene terephthalate, polyurethane, polyvinyl acetate, polyvinylidene chloride and polyethylene. In some embodiments, the separating layer comprises one or more polymers of polyesters, polyethylenes, polypropylenes, polyvinylchloride, polyethylene vinyl acetate or copolymers thereof, or polyurethanes. In one particular, but non-limiting embodiment, the separating layer is formed of a polyester film laminate. One particular polyester film laminate is the polyethylene and polyester laminate such as the laminate sold under the name SCOTCHPAK™ #9723. In some embodiments, the separating layer includes SCOTCHPAK™ #1012. In some embodiments, the separating layer includes SCOTCHPAK™ #9732.

In some embodiments, the separating layer comprises one or more polymers selected from polyesters, polyethylenes, polypropylenes, polystyrenes, polyvinylchloride, and a polyethylene terephthalate/ethylene vinyl acetate laminate. In some embodiments, the separating layer comprises polyester.

In some embodiments, the top surface of the separating layer is treated with a high-energy surface treatment. In some embodiments, the separating layer further comprises a coating of ethylene-vinyl acetate copolymer. In some embodiments, the top surface of the separating layer comprises the coating of ethylene-vinyl acetate copolymer.

In some embodiments, the high-energy surface treatment is selected from the group consisting of corona discharge treatment, plasma treatment, UV radiation, ion beam treatment, electron beam treatment and combinations thereof. In some embodiments, the high-energy surface treatment is corona discharge treatment.

In some embodiments, the top surface of the separating layer comprises a coating of ethylene-vinyl acetate copolymer treated with the high-energy surface treatment. In some embodiments, the top surface of the separating layer comprises a coating of ethylene-vinyl acetate copolymer treated with the corona discharge treatment. In some embodiments, the top surface of the separating layer comprises a coating of ethylene-vinyl acetate copolymer treated with the corona discharge treatment performed using a power of about 0.24 kW.

The top surface of the separating layer treated with the corona discharge treatment can have any suitable surface energy. For example, the top surface of the separating layer treated with the corona discharge treatment can have a surface energy of, but not limited to, at least 20 Dynes, or 25, 30, 35, 40, 45, 50, 55, 60, 65, or at least 70 Dynes. Alternatively, the top surface of the separating layer treated with the corona discharge treatment can have a surface energy of, but not limited to, at least 41 Dynes, or 42, 43, 44, 45, 46, 47, 48, 49, 50, 51, 52, 53, 54, 55, 56, 57, 58, 59, or at least 60 Dynes. In some embodiments, the top surface of the separating layer has a surface energy of at least 40 Dynes. The surface energy can be measured using a variety of techniques and instruments known to one of skill in the art, including, but not limited to, Mobile Surface Analyzer by Kruss, DyneTEC test kit from Tantec A/S, cotton-swab applicators, solution-tipped “dyne-pens”, and full-etch drawdown rods.

In some embodiments, the present invention provides a transdermal delivery system, comprising:
(1) a backing layer;
(2) a separating layer treated with a high-energy surface treatment, wherein the separating layer has a top surface and a bottom surface such that the top surface is in contact with the backing layer;
(3) a drug matrix layer comprising donepezil HCl and donepezil free base, wherein the drug matrix layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer,
   wherein the contact adhesive layer comprises donepezil free base in an amount of from 0.1 to 10% (w/w) of the total weight of the contact adhesive layer.

In some embodiments, the present invention provides a transdermal delivery system, comprising:
(1) a backing layer;
(2) a separating layer having a top surface and a bottom surface such that the top surface is in contact with the backing layer, wherein the top surface has a surface energy of at least 40 Dynes;
(3) a drug matrix layer comprising donepezil HCl and donepezil free base, wherein the drug matrix layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer,
   wherein the contact adhesive layer comprises donepezil free base in an amount of from 0.1 to 10% (w/w) of the total weight of the contact adhesive layer.

In some embodiments, the present invention provides a transdermal delivery system, comprising:

(1) a backing layer;

(2) a separating layer having a top surface and a bottom surface such that the top surface is in contact with the backing layer, wherein the top surface has a surface energy of at least 40 Dynes;

(3) a drug matrix layer comprising donepezil HCl, wherein the drug matrix layer has a top surface and a bottom surface such that the top surface is in contact with the bottom surface of the separating layer;

(4) a membrane layer comprising a microporous membrane, wherein the membrane layer has a top surface and a bottom surface such that the top surface is in contact with the bottom surface of the drug matrix layer; and

(5) a contact adhesive layer having a top surface and a bottom surface such that the top surface is in contact with the bottom surface of the membrane layer.

### Drug Matrix Layer

The transdermal delivery system also includes a drug matrix layer. The drug matrix layer includes donepezil HCl, and has a top surface and a bottom surface such that the top surface is in contact with the bottom surface of the separating layer.

The drug matrix layer can include the donepezil HCl in any suitable amount. For example, the drug matrix layer can include donepezil HCl in an amount of, but not limited to, from 1-50% (w/w), or 1-45%, 1-40%, 5-35%, 5-30%, 5-25%, 10-25%, 10-20%, 11-19%, 12-18%, 13-17%, or 14-16% (w/w). The drug matrix layer can also include donepezil HCl in an amount of, but not limited to, about 14.5% (w/w), or about 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, or about 16.5% (w/w). In some embodiments, the drug matrix layer can include donepezil HCl in an amount of 14-16% (w/w). In some embodiments, the drug matrix layer can include donepezil HCl in an amount of about 15% (w/w). In some embodiments, the drug matrix layer can include donepezil HCl in an amount of about 15.4% (w/w). In some embodiments, the drug matrix layer can include donepezil HCl in an amount of 15.4% (w/w). The weight percentages provided can represent the weight percentage of donepezil HCl to the total weight of the drug matrix layer.

Without being bound to any particular theory, the drug matrix solvent composition (i) enables the salt form of the active agent to be dissolved and/or suspended in the drug matrix layer, (ii) supports the in situ reaction of the salt form of the active agent to the base form of the active agent, and (iii) enables the base form of the active agent to be dissolved or solubilized in the drug matrix layer, for diffusion into the microporous membrane and into the contact adhesive layer.

The drug matrix layer can include a variety of other components. For example, other components include, but are not limited to, donepezil free base, an adhesive matrix, an acrylate polymer, a drug matrix solvent composition, an alkaline salt, and others.

In some embodiments, the drug matrix layer further comprises donepezil free base. The donepezil free base can be present in any suitable amount. For example, the drug matrix layer includes donepezil free base in an amount of, but not limited to, at least 1% (w/w) of the total weight of donepezil free base and donepezil hydrochloride, or at least 5, 10, 15, 20, 21, 22, 23, 24, 25, 26, 27, 28, 29, 30, or at least 35% (w/w). The drug matrix layer includes donepezil free base in an amount of, but not limited to, from 1 to 50% (w/w), or from 5 to 45% (w/w), or from 10 to 40% (w/w), or from 20 to 40% (w/w), or from 21 to 39% (w/w), or from 22 to 37% (w/w), or from 22 to 36% (w/w), or from 22 to 35% (w/w), or from 25 to 35% (w/w) of the total weight of donepezil free base and donepezil hydrochloride.

In some embodiments, the drug matrix layer is a composition comprising an adhesive matrix comprising an adhesive polymer, a drug matrix solvent composition and donepezil free base generated in situ in the drug matrix layer by reaction of a donepezil salt and an alkaline salt or another amphoteric base compound. The drug matrix layer is manufactured using a salt form of donepezil, e.g., donepezil hydrochloride (HCl), and an alkaline salt that react in situ to form donepezil free base.

In some embodiments, the drug matrix layer further comprises: (i) an acrylate copolymer, (ii) a drug matrix solvent composition comprising glycerin and one or more of lauryl lactate, sorbitan monolaurate and triethyl citrate, and (iv) an alkaline salt comprising sodium bicarbonate.

A drug matrix layer as described herein and hereinabove is contemplated for use in a transdermal delivery system, where the system additionally comprises an adhesive component. The adhesive component can be present in an amount of, but not limited to, about 50-90% (w/w) of adhesive polymer or copolymer, or between about 55-90% (w/w), or between about 60-90% (w/w), between about 65-90% (w/w), between about 70-90% (w/w), between about 75-90% (w/w), or between about 80-90% (w/w). The weight percentages provided can represent the weight percentage of adhesive polymer or copolymer to the total weight of the drug matrix layer. In some embodiments, the skin contact adhesive is comprised of a copolymer of acrylate/vinyl acetate. In some embodiments, the adhesive component additionally comprises a polyvinylpyrrolidone, such as a crosslinked polyvinylpyrrolidone.

The adhesive component in the drug matrix layer can be any of a variety of adhesive materials, such as pressure sensitive adhesive polymers. Polyacrylate pressure sensitive adhesive polymers are an example, and typically comprise a polyacrylate that is a polymer or a copolymer of a monomer or monomers selected from acrylic acid esters and methacrylic acid esters. Other monomers, such as acrylic acid and vinyl acetate, may be present. In some embodiments, the acrylic polymer is based on acrylic esters such as 2-ethylhexyl acrylate (2-EHA) and ethyl acrylate. In some embodiments, the polyacrylate polymer is a polymer or a copolymer of a monomer or monomers selected from acrylic acid and vinyl acetate. In some embodiments, the acrylic polymer adhesive has pendent carboxyl (—COOH) or hydroxyl (—OH) functional groups. In some embodiments, the acrylic polymer adhesive comprises at least one of polyacrylate, polymethacrylate, derivatives thereof, and co-polymers thereof. In some embodiments, the acrylic adhesive is comprised of an acrylate copolymer comprising acrylic ester monomers, acrylic acid, and/or vinyl acetate monomers. A copolymer of acrylic acid and vinyl acetate is one example. Acrylate copolymers are sold under the trade-name DURO-TAK® and include, but are not limited to, DURO-TAK 87-2287, 387-2516, 387-2051, and 387-2074. In some embodiments, the acrylate polymer comprises DURO-TAK 82-2287.

In some embodiments, the drug matrix layer comprises at least about 25-80% (w/w) of adhesive polymers relative to the weight of the drug matrix layer (inclusive of sub-ranges). In some embodiments, the drug matrix layer includes an adhesive polymer or copolymer or mixture of polymers and/or copolymers in an amount of, but not limited to, about 35-80%, 30-75%, at least about 40-75%, at least about 50-75%, at least about 60-75%, at least about 25-70%, at least about 30-70%, at least about 40-70%, at least about 50-70%, at least about 60-70%, at least about 25-60%, at least about 30-60%, at least about 40-60%, at least about 50-60%, at least about 25-50%, at least about 30-50%, at least about 40-50%, at least about 25-40%, at least about 30-40%, or at least about 25-30% (w/w). The drug matrix layer can include one or more or at least one adhesive polymers or copolymers. In some embodiments, the drug matrix layer includes at least about 5-75% of an individual polymer relative to the total weight of the polymers in the matrix. In some embodiments, the drug matrix layer includes an individual polymer in an amount of, but not limited to, about 5-10%, 5-15%, 5-20%, 5-25%, 5-30%, 5-40%, 5-50%, 5-60%, 5-70%, 5-75%, 10-15%, 10-20%, 10-20%, 10-25%, 10-30%, 10-40%, 10-50%, 10-60%, 10-70%, 10-75%, 15-20%, 15-25%, 15-30%, 15-40%, 15-50%, 15-60%, 15-70%, 15-75%, 20-25%, 20-30%, 20-40%, 20-50%, 20-60%, 20-70%, 20-75%, 25-30%, 25-40%, 25-50%, 25-60%, 25-70%, 25-75%, 30-40%, 30-50%, 30-60%, 30-70%, 30-75%, 40-50%, 40-60%, 40-70%, 40-75%, 50-60%, 50-70%, 50-75%, 60-70%, 60-75%, or 70-75% (w/w). In some embodiments, the drug matrix layer includes the acrylate polymer in an amount of from 30-50% (w/w). In some embodiments, the drug matrix layer includes the acrylate polymer in an amount of from 35-45% (w/w). In some embodiments, the drug matrix layer includes the acrylate polymer in an amount of from 37-41% (w/w). In some embodiments, the drug matrix layer includes the acrylate polymer in an amount of about 39% (w/w). In some embodiments, the drug matrix layer includes the acrylate polymer in an amount of about 39.1% (w/w). In some embodiments, the drug matrix layer includes the acrylate polymer in an amount of 39.1% (w/w). In some embodiments, the drug matrix layer includes the acrylate polymer in an amount of about 39.0% (w/w). In some embodiments, the drug matrix layer includes the acrylate polymer in an amount of 39.0% (w/w). In some embodiments, the drug matrix layer includes the acrylate polymer in an amount of about 38.9% (w/w). In some embodiments, the drug matrix layer includes the acrylate polymer in an amount of 38.9% (w/w). In some embodiments, the drug matrix layer includes the acrylate polymer in an amount of about 38.8% (w/w). In some embodiments, the drug matrix layer includes the acrylate polymer in an amount of 38.8% (w/w). In some embodiments, the drug matrix layer includes the acrylate polymer in an amount of about 38.7% (w/w). In some embodiments, the drug matrix layer includes the acrylate polymer in an amount of 38.7% (w/w). The weight percentages provided can represent the weight percentage of acrylate polymer to the total weight of the drug matrix layer.

In some embodiments, the drug matrix solvent composition and the membrane solvent composition have one, two, or three identical solvents. In some embodiments, the drug matrix solvent composition and the membrane solvent composition are comprised of the same solvents. For example, the drug matrix solvent composition and the membrane solvent composition each comprise a citrate ester, a surfactant, and/or an ester of α-hydroxy acid. In some embodiments, the drug matrix solvent composition (in the drug matrix layer) comprises a hydrophilic solvent that is excluded from, or is not present in, the membrane solvent composition or in the contact adhesive solvent composition.

In some embodiments, drug matrix solvent composition includes, but is not limited to, methyl laurate, propylene glycol monolaurate, glycerol monolaurate, glycerol monooleate, lauryl lactate, myristyl lactate, and dodecyl acetate. Additional drug matrix solvent compositions are described in U.S. Pat. No. 8,874,879, which is incorporated herein by reference. It will be appreciated that the compositions herein may include one or more or at least one drug matrix solvent composition.

The drug matrix layer also comprises a drug matrix solvent composition. In some embodiments, the drug matrix solvent composition includes one, two, three or four solvents. In some embodiments, the drug matrix solvent composition comprises triethyl citrate. In some embodiments, one or both of glycerine and sorbitan monolaurate are additionally present. In some embodiments, an ester of α-hydroxy acid as a further solvent in the drug matrix solvent composition is present. Exemplary esters of α-hydroxy acid solvents are esters of lactic acid or glycolic acid, and an example is lauryl lactate. In some embodiments, the drug matrix solvent composition is comprised of, consists essentially of, or consists of triethyl citrate, sorbitan monolaurate, lauryl lactate and glycerine.

In some embodiments, the drug matrix solvent composition can include a hydrophilic material or component that is not included in the membrane layer drug matrix solvent composition. In some embodiments, the hydrophilic material that is present in one or both of the contact adhesive layer and/or the drug matrix solvent composition but is not present in the membrane solvent composition is a hydrophilic solvent such as, but are not limited to, glycerine, water, and mixtures thereof. Other hydrophilic materials include, but are not limited to propylene glycols and low-weight polyethylene glycols. In some embodiments, the microporous membrane is a manufactured from a hydrophobic material to provide a hydrophobic microporous membrane; an example is a polypropylene microporous membrane or a polyethylene microporous membrane. Without being bound by any particular theory, a hydrophilic material, such as a hydrophilic solvent in the drug matrix solvent composition that is within the drug matrix layer does not diffuse or permeate into the microporous membrane or into the pores of the microporous membrane due to the hydrophobicity of the membrane material. The hydrophilic material in the drug matrix solvent composition within the drug matrix layer facilitates and supports the in situ formation of the water insoluble basic active agent from a pharmaceutically acceptable salt thereof. After the base form of the active agent is formed in the drug matrix layer, the base form of the active agent is solubilized by at least one component in the drug matrix solvent composition and by at least one component in the membrane layer drug matrix solvent composition, so that the base form of the active agent diffuses from the drug matrix layer into and through the hydrophobic pores of the microporous membrane. In some embodiments, the drug matrix solvent composition and the membrane solvent composition have one, two, or three identical solvents, yet the drug matrix solvent composition and the membrane solvent composition are different. For example, In some embodiments, the drug matrix solvent composition and the membrane solvent composition each comprise a citrate ester, a surfactant, and/or an α-hydroxy acid, and the drug matrix solvent composition comprises a hydrophilic solvent that is excluded from, or is not present in, the membrane layer drug matrix solvent composition.

In some embodiments, the drug matrix layer includes the drug matrix solvent composition in an amount of about 10-50% (w/w) of drug matrix solvent composition relative to the weight of the drug matrix layer (inclusive of sub-ranges). In some embodiments, the drug matrix layer includes the drug matrix solvent composition in an amount of, but not limited to, about 10-45%, 15-45%, 15-40%, 15-35%, 20-35%, 20-30%, or 25-30% (w/w). The drug matrix layer can also include the drug matrix solvent composition in an amount of, but not limited to, about 20% (w/w), or about 21, 22, 23, 24, 25, 26, 27, 28, 29, 30, 31, 32, 33, 34, or about 35% (w/w). In some embodiments, the drug matrix layer includes the drug matrix solvent composition in an amount of about 28% (w/w). In some embodiments, the drug matrix layer includes the drug matrix solvent composition in an amount of about 27.9% (w/w). In some embodiments, the drug matrix layer includes the drug matrix solvent composition in an amount of 27.9% (w/w). The weight percentages provided can represent the weight percentage of the drug matrix solvent composition to the total weight of the drug matrix layer.

In some embodiments, the drug matrix solvent composition of the drug matrix layer includes glycerine. The glycerine can be present in any suitable amount in the drug matrix layer. For example, the drug matrix layer can include glycerine in an amount of, but not limited to, about 1-20% (w/w), or about 2-19%, or about 3-18%, or about 4-17%, or about 5-16%, or about 5-15%, or about 6-15%, or about 7-15%, or about 8-14%, or about 9-13%, or about 10-12% (w/w). The drug matrix layer can also include glycerine in an amount of, but not limited to, about 5% (w/w), or about 6, 7, 8, 9, 10, 11, 12, 13, 14, or about 15% (w/w). In some embodiments, the drug matrix layer includes glycerine in an amount of about 11% (w/w). In some embodiments, the drug matrix layer includes glycerine in an amount of about 11.5% (w/w). In some embodiments, the drug matrix layer includes glycerine in an amount of 11.5% (w/w). The weight percentages provided can represent the weight percentage of glycerine to the total weight of the drug matrix layer.

In some embodiments, the drug matrix solvent composition of the drug matrix layer includes triethyl citrate. The triethyl citrate can be present in in any suitable amount in the drug matrix layer. For example, the drug matrix solvent composition of the drug matrix layer can include triethyl citrate in an amount of, but not limited to, about 1-20% (w/w), or about 2-19%, or about 3-18%, or about 4-17%, or about 5-16%, or about 5-15%, or about 6-15%, or about 7-15%, or about 8-14%, or about 9-13%, or about 10-12% (w/w). The drug matrix layer can also include triethyl citrate in an amount of, but not limited to, about 5% (w/w), or about 6, 7, 8, 9, 10, 11, 12, 13, 14, or about 15% (w/w). In some embodiments, the drug matrix layer includes triethyl citrate in an amount of about 11% (w/w). In some embodiments, the drug matrix layer includes triethyl citrate in an amount of about 11.2% (w/w). In some embodiments, the drug matrix layer includes triethyl citrate in an amount of 11.2% (w/w). The weight percentages provided can represent the weight percentage of triethyl citrate to the total weight of the drug matrix layer.

In some embodiments, the drug matrix solvent composition of the drug matrix layer includes lauryl lactate. The lauryl lactate can be present in any suitable amount in the drug matrix layer. For example, the drug matrix solvent composition of the drug matrix layer can include lauryl lactate in an amount of, but not limited to, about 0.1-10% (w/w), or about 0.5-10%, or about 1-10%, or about 1-5%, or about 2-4% (w/w). The drug matrix layer can also include lauryl lactate in an amount of, but not limited to, about 1% (w/w), or about 1.5, 2.0, 2.5, 3.0, 3.1, 3.2, 3.3, 3.4, 3.5, 3.6, 3.7, 3.8, 3.9, 4.0, 4.5, or about 5.0% (w/w). In some embodiments, the drug matrix layer includes lauryl lactate in an amount of about 3% (w/w). In some embodiments, the drug matrix layer includes lauryl lactate in an amount of about 3.3% (w/w). In some embodiments, the drug matrix layer includes lauryl lactate in an amount of 3.3% (w/w). The weight percentages provided can represent the weight percentage of lauryl lactate to the total weight of the drug matrix layer.

In some embodiments, the drug matrix solvent composition of the drug matrix layer includes sorbitan monolaurate. The sorbitan monolaurate can be present in any suitable amount in the drug matrix layer. For example, the drug matrix layer can include sorbitan monolaurate in an amount of, but not limited to, about 0.1-10% (w/w), or about 0.1-5%, or about 0.5-5%, or about 1-5%, or about 1-3% (w/w). The drug matrix layer can also include sorbitan monolaurate in an amount of, but not limited to, about 1% (w/w), or about 1.1, 1.2, 1.3, 1.4, 1.5, 1.6, 1.7, 1.8, 1.9, 2.0, 2.1, 2.2, 2.3, 2.4, or about 2.5% (w/w). In some embodiments, the drug matrix layer includes sorbitan monolaurate in an amount of about 2% (w/w). In some embodiments, the drug matrix layer includes sorbitan monolaurate in an amount of about 1.9% (w/w). In some embodiments, the drug matrix layer includes sorbitan monolaurate in an amount of 1.9% (w/w). The weight percentages provided can represent the weight percentage of sorbitan monolaurate to the total weight of the drug matrix layer.

The alkaline salt can be, for example, sodium bicarbonate, sodium carbonate, potassium carbonate, potassium bicarbonate, trisodium phosphate, disodium hydrogen phosphate, sodium oxylate, sodium succinate, sodium citrate, or sodium salicylate. In some embodiments, the alkaline salt includes sodium bicarbonate. In some embodiments, the alkaline salt consists essentially of sodium bicarbonate. In some embodiments, the alkaline salt consists of sodium bicarbonate.

In some embodiments, the present invention provides a transdermal delivery system, comprising:
(1) a backing layer;
(2) a separating layer having a top surface and a bottom surface such that the top surface is in contact with the backing layer;
(3) a drug matrix layer comprising donepezil HCl, donepezil free base, and sodium bicarbonate, wherein the drug matrix layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer.

In some embodiments, the present invention provides a transdermal delivery system, comprising:
(1) a backing layer;
(2) a separating layer having a top surface and a bottom surface such that the top surface is in contact with the backing layer;
(3) a drug matrix layer comprising donepezil HCl, donepezil free base, and sodium bicarbonate particles having a D90 particle size of from 1 μm to 500 μm, wherein the drug matrix layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer.

In some embodiments, the present invention provides a transdermal delivery system, comprising:
(1) a backing layer;
(2) a separating layer treated with a high-energy surface treatment, wherein the separating layer has a top surface and a bottom surface such that the top surface is in contact with the backing layer;
(3) a drug matrix layer comprising donepezil HCl, donepezil free base, and sodium bicarbonate, wherein the drug matrix layer has a top surface and a bottom surface such that the top surface is in contact with the bottom surface of the separating layer;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer, wherein the contact adhesive layer comprises donepezil free base in an amount of from 0.1 to 10% (w/w) of the total weight of the contact adhesive layer.

The sodium bicarbonate can be in any suitable particle size. For example, the sodium bicarbonate can include, but is not limited to, particles having a D90 particle size of, but not limited to, from 0.1 μm to 1000 μm, or from 0.1 μm to 900 μm, or from 0.1 μm to 800 μm, or from 0.1 μm to 700 μm, or from 0.1 μm to 600 μm, or from 0.1 μm to 500 μm, or from 0.1 μm to 400 μm, or from 0.1 μm to 300 μm, or from 0.1 μm to 200 μm, or from 0.1 μm to 100 μm, or from 0.1 μm to 90 μm, or from 0.1 μm to 85 μm, or from 0.1 μm to 80 μm, or from 0.1 μm to 75 μm, or from 0.1 μm to 70 μm, or from 0.1 μm to 65 μm, or from 0.1 μm to 60 μm, or from 0.1 μm to 65 μm, or from 0.1 μm to 60 μm, or from 0.1 μm to 55 μm, or from 0.1 μm to 50 μm, or from 0.1 μm to 45 μm, or from 0.1 μm to 40 μm, or from 0.1 μm to 35 μm, or from 0.1 μm to 30 μm, or from 0.1 μm to 25 μm, or from 0.1 μm to 20 μm, or from 0.1 μm to 15 μm, or from 0.1 μm to 10 μm. The sodium bicarbonate can include, but is not limited to, particles having a D90 particle size of, but not limited to, from 1 μm to 1000 μm, from 1 μm to 500 μm, from 1 μm to 200 μm, or from 1 μm to 100 μm, or from 1 μm to 90 μm, or from 1 μm to 85 μm, or from 1 μm to 80 μm, or from 1 μm to 75 μm, or from 1 μm to 70 μm, or from 1 μm to 65 μm, or from 1 μm to 60 μm, or from 1 μm to 65 μm, or from 1 μm to 60 μm, or from 1 μm to 55 μm, or from 1 μm to 50 μm, or from 1 μm to 45 μm, or from 1 μm to 40 μm, or from 1 μm to 35 μm, or from 1 μm to 30 μm, or from 1 μm to 25 μm, or from 1 μm to 20 μm, or from 1 μm to 15 μm, or from 1 μm to 10 μm. The sodium bicarbonate can include, but is not limited to, particles having a D90 particle size of, but not limited to, from 20 μm to 100 μm, or from 10 μm to 200 μm, or from 5 μm to 300 μm.

In some embodiments, the sodium bicarbonate comprises particles having a D90 particle size of from 0.1 μm to 1000 μm. In some embodiments, the sodium bicarbonate comprises particles having a D90 particle size of from 0.1 μm to 200 μm. In some embodiments, the sodium bicarbonate comprises particles having a D90 particle size of from 0.1 μm to 100 μm. In some embodiments, the sodium bicarbonate comprises particles having a D90 particle size of from 10 μm to 200 μm. In some embodiments, the sodium bicarbonate comprises particles having a D90 particle size of from 20 μm to 100 μm. In some embodiments, the sodium bicarbonate comprises particles having a D90 particle size of from 0.1 μm to 20 μm.

The alkaline salt can be present in various amounts. For example, the alkaline salt can be present in an amount of, but not limited to, about 0.1-10% (w/w), or about 0.1-5%, or about 0.5-5%, or about 1-5%, or about 2-4% (w/w), or about 2-3% (w/w). Alternatively, the alkaline salt is present in an amount of, but not limited to, about 2% (w/w), or about 2.1, 2.2, 2.3, 2.4, 2.5, 2.6, 2.7, 2.8, 2.9, 3.0, 3.1, 3.2, 3.3, 3.4, or about 3.5% (w/w). In some embodiments, the alkaline salt is present in an amount of about 2.7% (w/w). In some embodiments, the alkaline salt is present in an amount of 2.7% (w/w). In some embodiments, the alkaline salt is present in an amount of about 2.8% (w/w). In some embodiments, the alkaline salt is present in an amount of 2.8% (w/w). In some embodiments, the alkaline salt is present in an amount of about 2.9% (w/w). In some embodiments, the alkaline salt is present in an amount of 2.9% (w/w). In some embodiments, the alkaline salt is present in an amount of about 3.0% (w/w). In some embodiments, the alkaline salt is present in an amount of 3.0% (w/w). In some embodiments, the alkaline salt is present in an amount of about 3.1% (w/w). In some embodiments, the alkaline salt is present in an amount of 3.1% (w/w). The weight percentages provided can represent the weight percentage of the alkaline salt to the total weight of the drug matrix layer.

The sodium bicarbonate can be present in various amounts. For example, the sodium bicarbonate can be present in an amount of, but not limited to, about 0.1-10% (w/w), or about 0.1-5%, or about 0.5-5%, or about 1-5%, or about 2-4% (w/w), or about 2-3% 30 (w/w). Alternatively, the sodium bicarbonate is present in an amount of, but not limited to, about 2% (w/w), or about 2.1, 2.2, 2.3, 2.4, 2.5, 2.6, 2.7, 2.8, 2.9, 3.0, 3.1, 3.2, 3.3, 3.4, or about 3.5% (w/w). In some embodiments, the sodium bicarbonate is present in an amount of about 2.7% (w/w). In some embodiments, the sodium bicarbonate is present in an amount of 2.7% (w/w). In some embodiments, the sodium bicarbonate is present in an amount of about 2.8% (w/w). In some embodiments, the sodium bicarbonate is present in an amount of 2.8% (w/w). In some embodiments, the sodium bicarbonate is present in an amount of about 2.9% (w/w). In some embodiments, the sodium bicarbonate is present in an amount of 2.9% (w/w). In some embodiments, the sodium bicarbonate is present in an amount of about 3.0% (w/w). In some embodiments, the sodium bicarbonate is present in an amount of 3.0% (w/w). In some embodiments, the sodium bicarbonate is present in an amount of about 3.1% (w/w). In some embodiments, the drug matrix layer includes sodium bicarbonate in an amount of about 3.1% (w/w). In some embodiments, the drug matrix layer includes sodium bicarbonate in an amount of 3.1% (w/w). The weight percentages provided can represent the weight percentage of the sodium bicarbonate to the total weight of the drug matrix layer.

The sodium bicarbonate can be present in any suitable molar ratio to the donepezil HCl. For example, the sodium bicarbonate can be present in a molar ratio of 1.0 or less, including 0.5 to 1.0, 0.6 to 1.0, 0.7 to 1.0, 0.8 to 1.0, 0.85 to 1.0, 0.9 to 1.0, or 0.95 to 1.0. In some embodiments, the sodium bicarbonate can be present in a molar ratio to the donepezil HCl of about 0.5, 0.55, 0.6, 0.65, 0.7, 0.75, 0.8, 0.85, 0.86, 0.87, 0.88, 0.89, 0.90, 0.91, 0.92, 0.93, 0.94, 0.95, 0.96, 0.97, 0.98, 0.99, or about 1.0. In some embodiments, the sodium bicarbonate can be present in a molar ratio to the donepezil HCl of about 0.88. In some embodiments, the sodium bicarbonate can be present in a molar ratio to the donepezil HCl of about 0.92. In some embodiments, the sodium bicarbonate can be present in a molar ratio to the donepezil HCl of about 0.95. In some embodiments, the sodium bicarbonate can be present in a molar ratio to the donepezil HCl of about 1.0.

In some embodiments, the drug matrix layer includes sodium bicarbonate in an amount of about 2.7% (w/w), wherein the sodium bicarbonate comprises particles having a D90 particle size of from 1 μm to 500 μm. In some embodiments, the drug matrix layer includes sodium bicarbonate in an amount of about 2.7% (w/w), wherein the sodium bicarbonate comprises particles having a D90 particle size of from 0.1 μm to 200 μm. In some embodiments, the drug matrix layer includes sodium bicarbonate in an amount of about 2.7% (w/w), wherein the sodium bicarbonate comprises particles having a D90 particle size of from 0.1 μm to 100 μm. In some embodiments, the drug matrix layer includes sodium bicarbonate in an amount of about 2.7% (w/w), wherein the sodium bicarbonate comprises particles having a D90 particle size of from 0.1 μm to 20 μm. In some embodiments, the drug matrix layer includes sodium bicarbonate in an amount of 2.7% (w/w), wherein the sodium bicarbonate comprises particles having a D90 particle size of from 0.1 μm to 20 μm. The weight percentages provided can represent the weight percentage of the sodium bicarbonate to the total weight of the drug matrix layer. In some embodiments, the drug matrix layer includes sodium bicarbonate in an amount of about 2.7% (w/w), wherein the sodium bicarbonate comprises particles having a D90 particle size of from 20 μm to 100 μm. In some embodiments, the drug matrix layer includes sodium bicarbonate in an amount of 2.7% (w/w), wherein the sodium bicarbonate comprises particles having a D90 particle size of from 20 μm to 100 μm. The weight percentages provided can represent the weight percentage of the sodium bicarbonate to the total weight of the drug matrix layer.

In some embodiments, the drug matrix layer includes sodium bicarbonate in an amount of about 2.8% (w/w), wherein the sodium bicarbonate comprises particles having a D90 particle size of from 1 μm to 500 μm. In some embodiments, the drug matrix layer includes sodium bicarbonate in an amount of about 2.8% (w/w), wherein the sodium bicarbonate comprises particles having a D90 particle size of from 0.1 μm to 200 μm. In some embodiments, the drug matrix layer includes sodium bicarbonate in an amount of about 2.8% (w/w), wherein the sodium bicarbonate comprises particles having a D90 particle size of from 0.1 μm to 100 μm. In some embodiments, the drug matrix layer includes sodium bicarbonate in an amount of about 2.8% (w/w), wherein the sodium bicarbonate comprises particles having a D90 particle size of from 0.1 μm to 20 μm. In some embodiments, the drug matrix layer includes sodium bicarbonate in an amount of 2.8% (w/w), wherein the sodium bicarbonate comprises particles having a D90 particle size of from 0.1 μm to 20 μm. The weight percentages provided can represent the weight percentage of the sodium bicarbonate to the total weight of the drug matrix layer. In some embodiments, the drug matrix layer includes sodium bicarbonate in an amount of about 2.8% (w/w), wherein the sodium bicarbonate comprises particles having a D90 particle size of from 20 μm to 100 μm. In some embodiments, the drug matrix layer includes sodium bicarbonate in an amount of 2.8% (w/w), wherein the sodium bicarbonate comprises particles having a D90 particle size of from 20 μm to 100 μm. The weight percentages provided can represent the weight percentage of the sodium bicarbonate to the total weight of the drug matrix layer.

In some embodiments, the drug matrix layer includes sodium bicarbonate in an amount of about 2.9% (w/w), wherein the sodium bicarbonate comprises particles having a D90 particle size of from 1 μm to 500 μm. In some embodiments, the drug matrix layer includes sodium bicarbonate in an amount of about 2.9% (w/w), wherein the sodium bicarbonate comprises particles having a D90 particle size of from 0.1 μm to 200 μm. In some embodiments, the drug matrix layer includes sodium bicarbonate in an amount of about 2.9% (w/w), wherein the sodium bicarbonate comprises particles having a D90 particle size of from 0.1 μm to 100 μm. In some embodiments, the drug matrix layer includes sodium bicarbonate in an amount of about 2.9% (w/w), wherein the sodium bicarbonate comprises particles having a D90 particle size of from 0.1 μm to 20 μm. In some embodiments, the drug matrix layer includes sodium bicarbonate in an amount of 2.9% (w/w), wherein the sodium bicarbonate comprises particles having a D90 particle size of from 0.1 μm to 20 μm. The weight percentages provided can represent the weight percentage of the sodium bicarbonate to the total weight of the drug matrix layer. In some embodiments, the drug matrix layer includes sodium bicarbonate in an amount of about 2.9% (w/w), wherein the sodium bicarbonate comprises particles having a D90 particle size of from 20 μm to 100 μm. In some embodiments, the drug matrix layer includes sodium bicarbonate in an amount of 2.9% (w/w), wherein the sodium bicarbonate comprises particles having a D90 particle size of from 20 μm to 100 μm. The weight percentages provided can represent the weight percentage of the sodium bicarbonate to the total weight of the drug matrix layer.

In some embodiments, the drug matrix layer includes sodium bicarbonate in an amount of about 3.0% (w/w), wherein the sodium bicarbonate comprises particles having a D90 particle size of from 1 μm to 500 μm. In some embodiments, the drug matrix layer includes sodium bicarbonate in an amount of about 3.0% (w/w), wherein the sodium bicarbonate comprises particles having a D90 particle size of from 0.1 μm to 200 μm. In some embodiments, the drug matrix layer includes sodium bicarbonate in an amount of about 3.0% (w/w), wherein the sodium bicarbonate comprises particles having a D90 particle size of from 0.1 μm to 100 μm. In some embodiments, the drug matrix layer includes sodium bicarbonate in an amount of about 3.0% (w/w), wherein the sodium bicarbonate comprises particles having a D90 particle size of from 0.1 μm to 20 μm. In some embodiments, the drug matrix layer includes sodium bicarbonate in an amount of 3.0% (w/w), wherein the sodium bicarbonate comprises particles having a D90 particle size of from 0.1 μm to 20 μm. The weight percentages provided can represent the weight percentage of the sodium bicarbonate to the total weight of the drug matrix layer. In some embodiments, the drug matrix layer includes sodium bicarbonate in an amount of about 3.0% (w/w), wherein the sodium bicarbonate comprises particles having a D90 particle size of from 20 μm to 100 μm. In some embodiments, the drug matrix layer includes sodium bicarbonate in an amount of 3.0% (w/w), wherein the sodium bicarbonate comprises particles having a D90 particle size of from 20 μm to 100 μm. The weight percentages provided can represent the weight percentage of the sodium bicarbonate to the total weight of the drug matrix layer.

In some embodiments, the drug matrix layer includes sodium bicarbonate in an amount of about 3.1% (w/w), wherein the sodium bicarbonate comprises particles having a D90 particle size of from 1 μm to 500 μm. In some embodiments, the drug matrix layer includes sodium bicarbonate in an amount of about 3.1% (w/w), wherein the sodium bicarbonate comprises particles having a D90 particle size of from 0.1 μm to 200 μm. In some embodiments, the drug matrix layer includes sodium bicarbonate in an amount of about 3.1% (w/w), wherein the sodium bicarbonate comprises particles having a D90 particle size of from 0.1 μm to 100 μm. In some embodiments, the drug matrix layer includes sodium bicarbonate in an amount of about 3.1% (w/w), wherein the sodium bicarbonate comprises particles having a D90 particle size of from 0.1 μm to 20 μm. In some embodiments, the drug matrix layer includes sodium bicarbonate in an amount of 3.1% (w/w), wherein the sodium bicarbonate comprises particles having a D90 particle size of from 0.1 μm to 20 μm. The weight percentages provided can represent the weight percentage of the sodium bicarbonate to the total weight of the drug matrix layer. In some embodiments, the drug matrix layer includes sodium bicarbonate in an amount of about 3.1% (w/w), wherein the sodium bicarbonate comprises particles having a D90 particle size of from 20 μm to 100 μm. In some embodiments, the drug matrix layer includes sodium bicarbonate in an amount of 3.1% (w/w), wherein the sodium bicarbonate comprises particles having a D90 particle size of from 20 μm to 100 μm. The weight percentages provided can represent the weight percentage of the sodium bicarbonate to the total weight of the drug matrix layer.

The drug matrix layer can include the donepezil HCl and sodium bicarbonate in any suitable amounts. In some embodiments, the drug matrix layer includes donepezil HCl in an amount of 10-20% (w/w), and sodium bicarbonate in an amount of 1-5% (w/w), wherein the sodium bicarbonate comprises particles having a D90 particle size of from 1 μm to 500 μm. In some embodiments, the drug matrix layer includes donepezil HCl in an amount of 14-16% (w/w), and sodium bicarbonate in an amount of 2-4% (w/w), wherein the sodium bicarbonate comprises particles having a D90 particle size of from 10 μm to 200 μm.

In some embodiments, the drug matrix layer includes donepezil HCl in an amount of about 15% (w/w), and sodium bicarbonate in an amount of about 2.7% (w/w), wherein the sodium bicarbonate comprises particles having a D90 particle size of from 20 μm to 100 μm. In some embodiments, the drug matrix layer includes donepezil HCl in an amount of about 15.4% (w/w), and sodium bicarbonate in an amount of about 2.7% (w/w), wherein the sodium bicarbonate comprises particles having a D90 particle size of from 20 μm to 100 μm. In some embodiments, the drug matrix layer includes donepezil HCl in an amount of 15.4% (w/w), and sodium bicarbonate in an amount of 2.7% (w/w), wherein the sodium bicarbonate comprises particles having a D90 particle size of from 20 μm to 100 μm. The weight percentages provided can represent the weight percentage of donepezil HCl to the total weight of the drug matrix layer.

In some embodiments, the drug matrix layer includes donepezil HCl in an amount of about 15% (w/w), and sodium bicarbonate in an amount of about 2.8% (w/w), wherein the sodium bicarbonate comprises particles having a D90 particle size of from 20 μm to 100 μm. In some embodiments, the drug matrix layer includes donepezil HCl in an amount of about 15.4% (w/w), and sodium bicarbonate in an amount of about 2.8% (w/w), wherein the sodium bicarbonate comprises particles having a D90 particle size of from 20 μm to 100 μm. In some embodiments, the drug matrix layer includes donepezil HCl in an amount of 15.4% (w/w), and sodium bicarbonate in an amount of 2.8% (w/w), wherein the sodium bicarbonate comprises particles having a D90 particle size of from 20 μm to 100 μm. The weight percentages provided can represent the weight percentage of donepezil HCl to the total weight of the drug matrix layer.

In some embodiments, the drug matrix layer includes donepezil HCl in an amount of about 15% (w/w), and sodium bicarbonate in an amount of about 2.9% (w/w), wherein the sodium bicarbonate comprises particles having a D90 particle size of from 20 μm to 100 μm. In some embodiments, the drug matrix layer includes donepezil HCl in an amount of about 15.4% (w/w), and sodium bicarbonate in an amount of about 2.9% (w/w), wherein the sodium bicarbonate comprises particles having a D90 particle size of from 20 μm to 100 μm. In some embodiments, the drug matrix layer includes donepezil HCl in an amount of 15.4% (w/w), and sodium bicarbonate in an amount of 2.9% (w/w), wherein the sodium bicarbonate comprises particles having a D90 particle size of from 20 μm to 100 μm. The weight percentages provided can represent the weight percentage of donepezil HCl to the total weight of the drug matrix layer.

In some embodiments, the drug matrix layer includes donepezil HCl in an amount of about 15% (w/w), and sodium bicarbonate in an amount of about 3.0% (w/w), wherein the sodium bicarbonate comprises particles having a D90 particle size of from 20 μm to 100 μm. In some embodiments, the drug matrix layer includes donepezil HCl in an amount of about 15.4% (w/w), and sodium bicarbonate in an amount of about 3.0% (w/w), wherein the sodium bicarbonate comprises particles having a D90 particle size of from 20 μm to 100 μm. In some embodiments, the drug matrix layer includes donepezil HCl in an amount of 15.4% (w/w), and sodium bicarbonate in an amount of 3.0% (w/w), wherein the sodium bicarbonate comprises particles having a D90 particle size of from 20 μm to 100 μm. The weight percentages provided can represent the weight percentage of donepezil HCl to the total weight of the drug matrix layer.

In some embodiments, the drug matrix layer includes donepezil HCl in an amount of about 15% (w/w), and sodium bicarbonate in an amount of about 3.1% (w/w), wherein the sodium bicarbonate comprises particles having a D90 particle size of from 20 μm to 100 μm. In some embodiments, the drug matrix layer includes donepezil HCl in an amount of about 15.4% (w/w), and sodium bicarbonate in an amount of about 3.1% (w/w), wherein the sodium bicarbonate comprises particles having a D90 particle size of from 20 μm to 100 μm. In some embodiments, the drug matrix layer includes donepezil HCl in an amount of 15.4% (w/w), and sodium bicarbonate in an amount of 3.1% (w/w), wherein the sodium bicarbonate comprises particles having a D90 particle size of from 20 μm to 100 μm. The weight percentages provided can represent the weight percentage of donepezil HCl to the total weight of the drug matrix layer.

The drug matrix layer may further include one or more matrix modifiers. Without wishing to be bound by theory, it is believed that the matrix modifier facilitates homogenization of the adhesive matrix. Sorption of hydrophilic moieties is a possible mechanism for this process. Thus, known matrix modifiers which are to some degree water-sorbent may be used. For example, possible matrix modifiers include colloidal silicone dioxide, fumed silica, cross-linked polyvinylpyrrolidone (PVP), soluble PVP, cellulose derivatives (e.g. hydroxypropyl cellulose (HPC), hydroxyethylcellulose (HEC)), polyacrylamide, polyacrylic acid, polyacrylate, a polyacrylic acid salt, or a clay such as kaolin or bentonite. An exemplary commercial fumed silica product is Cab-O-Sil (Cabot Corporation, Boston, Mass.). The hydrophilic mixtures described in U.S. Published Patent Application No. 2003/0170308 may also be employed, for example mixtures of PVP and PEG or of PVP, PEG, and a water-swellable polymer such as EUDRAGIT® L100-55. In some embodiments, the matrix modifier is individually included in an amount between about 1-25%, about 2-25%, about 5-25%, about 5-7%, about 7-20%, or about 7-25% relative to the weight of the adhesive matrix (inclusive of sub-ranges). In some embodiments, the matrix modifier does not include ethylcellulose.

The drug matrix layer may also comprise a copolymer such as a polyvinylpyrrolidone/vinyl acetate copolymer, an acrylate/vinyl acetate copolymer, or a vinyl acetate/ethylene acetate copolymer. In some embodiments, the copolymer is a vinyl acetate/N-vinylpyrrolidone copolymer such as the copolymer sold as Plasdone™ S630 (Ashland). In some embodiments, the polyvinylpyrrolidone-vinyl acetate copolymer is a linear random copolymer of n-vinyl-2-pyrrolidone and vinyl acetate. In some embodiments, the copolymer is a 60:40 copolymer of n-vinyl-2-pyrrolidone and vinyl acetate.

The drug matrix layer may also comprise a polyvinylpyrrolidone (PVP). PVP is a water-soluble polymer comprised of the N-vinylpyrrolidone monomer, and is available in various forms, including cross-linked and non-crosslinked. In some of the working examples herein, a cross-linked PVP is included in the drug matrix layer. In some embodiments, the cross-linked PVP is Crospovidone. In some embodiments, the drug matrix layer further comprises Crospovidone.

The Crospovidone can be present in the drug matrix layer in any suitable amount. For example, the Crospovidone be present in the drug matrix layer in an amount of, but not limited to, from 1-50% (w/w), or 5-25%, or 10-20%, or 11-19%, or 12-18%, or 13-17%, or 14-16% (w/w). The drug matrix layer can also include Crospovidone in an amount of, but not limited to, about 13.5% (w/w), or about 13.6, 13.7, 13.8, 13.9, 14.0, 14.1, 14.2, 14.3, 14.4., 14.5, 14.6, 14.7, 14.8, 14.9, 15.0, 15.1, 15.2, 15.3, 15.4, or about 15.5% (w/w). In some embodiments, the drug matrix layer includes Crospovidone in an amount of about 14% (w/w). In some embodiments, the drug matrix layer includes Crospovidone in an amount of from 14 to 16% (w/w). In some embodiments, the drug matrix layer includes Crospovidone in an amount of about 14.4% (w/w). In some embodiments, the drug matrix layer includes Crospovidone in an amount of 14.4% (w/w). The weight percentages provided can represent the weight percentage of Crospovidone to the total weight of the drug matrix layer.

The drug matrix layer may further include other conventional additives such as adhesive agents, antioxidants, crosslinking or curing agents, pH regulators, pigments, dyes, refractive particles, conductive species, antimicrobial agents, opacifiers, gelling agents, viscosity modifiers or thickening agents, stabilizing agents, and the like as known in the art. In those embodiments wherein adhesion needs to be reduced or eliminated, conventional detackifying agents may also be used. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. These additives, and amounts thereof, are selected in such a way that they do not significantly interfere with the desired chemical and physical properties of the adhesive and/or active agent.

The drug matrix layer can also contain irritation-mitigating additives to minimize or eliminate the possibility of skin irritation and/or skin damage resulting from the drug, the enhancer, or other components of the composition. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; chloroquine; and corticosteriods.

In some embodiments, the drug matrix layer also includes an ascorbate. Any suitable ascorbate can be used in the transdermal delivery system of the present invention. Representative ascorbates include, but are not limited to, ascorbyl palmitate and ascorbyl stearate. In some embodiments, the drug matrix layer includes ascorbyl palmitate.

The drug matrix layer can include any suitable amount of ascorbyl palmitate. For example, the drug matrix layer can include the ascorbyl palmitate in an amount of, but not limited to, 0.01 to 10% (w/w), or 0.1 to 5%, or 0.1 to 4%, or 0.1 to 3%, or 0.1 to 2%, or 0.1 to 1%, or 0.2 to 0.9%, or 0.3 to 0.8%, or 0.4 to 0.6% (w/w). The drug matrix layer can also include the ascorbyl palmitate in an amount of, but not limited to, about 0.1% (w/w), or 0.2, 0.3, 0.4, 0.5, 0.6, 0.7, 0.8, 0.9, or about 1.0% (w/w). In some embodiments, the drug matrix layer includes ascorbyl palmitate in an amount of from 0.1 to 1.0% (w/w). In some embodiments, the drug matrix layer includes ascorbyl palmitate in an amount of from 0.4 to 0.6% (w/w). In some embodiments, the drug matrix layer includes ascorbyl palmitate in an amount of about 0.5% (w/w). In some embodiments, the drug matrix layer includes ascorbyl palmitate in an amount of 0.5% (w/w). The weight percentages provided can represent the weight percentage of ascorbyl palmitate to the total weight of the drug matrix layer.

In some embodiments, the drug matrix layer further comprises acrylate-vinyl acetate copolymer, glycerin, lauryl lactate, sorbitan monolaurate, triethyl citrate, donepezil free base, and sodium bicarbonate.

In some embodiments, the transdermal delivery system includes a drug matrix layer that comprises or consists essentially of donepezil free base, donepezil HCl and sodium bicarbonate; a drug matrix solvent composition mixture of triethyl citrate, sorbitan monolaurate, and glycerine; and a polymeric, adhesive matrix of crosslinked polyvinylpyrrolidone and a copolymer of acrylate/vinyl acetate is contemplated. In some embodiments, the drug matrix layer comprises or consists essentially of donepezil free base, about 10-25% (w/w) donepezil HCl and about 1-5% (w/w) sodium bicarbonate; about 5-15% (w/w) triethyl citrate; about 0.5-5% (w/w) sorbitan monolaurate; about 5-15% (w/w) glycerine; about 5-25% (w/w) crosslinked polyvinylpyrrolidone; and about 30-50% (w/w) acrylate-vinylacetate copolymer. The weight percentages provided can represent the weight percentage of each component to the total weight of the drug matrix layer.

In some embodiments, the transdermal delivery system includes a composition comprising a drug matrix layer consisting essentially of donepezil free base, about 14-18% (w/w) donepezil HCl and about 2-5% (w/w) sodium bicarbonate; about 8-12% (w/w) triethyl citrate; about 1.5-2.5% (w/w) sorbitan monolaurate; about 10-12% (w/w) glycerine; about 13-17% (w/w) crosslinked polyvinylpyrrolidone; and about 38-40% (w/w) acrylate-vinylacetate copolymer. The weight percentages provided can represent the weight percentage of each component to the total weight of the drug matrix layer.

The transdermal delivery system having a therapeutic agent can include a separating layer having any components as described within. In some embodiments, the separating layer comprises at least one of an occlusive material or a breathable material. In some embodiments, the separating layer comprises an occlusive material. In some embodiments, the separating layer comprises one or more polymers selected from polyesters, polyethylenes, polypropylenes, polystyrenes, polyvinylchloride, and a polyethylene terephthalate/ethylene vinyl acetate laminate. In some embodiments, the separating layer comprises a polyester polymer.

The transdermal delivery system having a therapeutic agent can include a top surface having any components as described within. In some embodiments, the top surface of the separating layer comprises a coating of ethylene-vinyl acetate copolymer treated with the high-energy surface treatment.

The transdermal delivery system having a therapeutic agent can include a high-energy surface treatment having any treatment described within. In some embodiments, the high-energy surface treatment is selected from the group consisting of corona discharge treatment, plasma treatment, UV radiation, ion beam treatment, electron beam treatment and combinations thereof. In some embodiments, the high-energy surface treatment is corona discharge treatment. In some embodiments, the top surface of the separating layer has a surface energy of at least 40 Dynes.

The transdermal delivery system having a therapeutic agent can include a drug matrix layer having any combination of components described within. The therapeutic agent can include any suitable therapeutic agent. For example, the therapeutic agent can include donepezil hydrochloride, donepezil free base, memantine, or combinations thereof.

In some embodiments, the drug matrix layer further comprises: (i) an acrylate copolymer, and (ii) a drug matrix solvent composition comprising glycerin and one or more of lauryl lactate, sorbitan monolaurate and triethyl citrate. In some embodiments, the drug matrix layer further comprises acrylate-vinyl acetate copolymer, glycerin, lauryl lactate, sorbitan monolaurate, and triethyl citrate. In some embodiments, the drug matrix layer further comprises ascorbyl palmitate.

The transdermal delivery system having a therapeutic agent can include a microporous membrane layer having any combination of components described within. In some embodiments, the microporous membrane comprises polypropylene. In some embodiments, the microporous membrane comprises a plurality of pores. In some embodiments, the plurality of pores in the microporous membrane contain a solvent composition comprised of one or more of triethyl citrate, sorbitan monolaurate, and lauryl lactate. In some embodiments, the microporous membrane comprises polypropylene, and the plurality of pores in the microporous membrane comprises triethyl citrate, sorbitan monolaurate, and lauryl lactate.

The transdermal delivery system having a therapeutic agent can include a contact adhesive layer having any combination of components described within. In some embodiments, the contact adhesive layer comprises a copolymer of acrylate and vinyl acetate. In some embodiments, the contact adhesive layer further comprises one or more solvents of triethyl citrate, sorbitan monolaurate, or lauryl lactate.

The transdermal delivery system having a therapeutic agent can include a release layer having any combination of components described within. In some embodiments, the transdermal delivery system also includes a release layer in contact with the bottom surface of the contact adhesive layer. In some embodiments, the release layer comprises a silicone coated material, a fluorocarbon coated material, or a fluorosilicone coated material. In some embodiments, the release layer comprises a silicone coated material.

The drug matrix layer can include any additional components as described within. In some embodiments, the drug matrix layer further comprises at least one of an acrylate polymer, glycerin, ascorbyl palmitate, lauryl lactate, sorbitan monolaurate and triethyl citrate.

The drug matrix layer can be prepared by the methods described herein.

The drug matrix layer also includes any combination of components described within.

### Membrane Layer (Intermediate Layer)

The membrane layer, also referred to as a fabric layer, an intermediate or a tie layer, may be formed of any suitable material including, but not limited to, polyesters, vinyl acetate polymers and copolymers, polyethylenes, and combinations thereof. In some embodiments, the membrane layer is a nonwoven layer of polyester fibers such as the film sold under the name Reemay® (Kavon Filter Products Co.). In some embodiments, the membrane layer does not affect the rate of release of the active agent from the adhesive layers.

In some embodiments, the membrane layer comprises a microporous membrane. For example, the microporous membrane can be a microporous polypropylene or polyethylene. The microporous membrane can help to control the rate of drug release from the transdermal delivery system. Several different microporous membranes are commercially available such as those sold under the name Celgard®, for example the Celgard® 2400 (Polypore International, LP).

Other materials useful in forming the microporous membrane include, but are not limited to polycarbonates, i.e., linear polyesters of carbonic acids in which carbonate groups recur in the polymer chain, by phosgenation of a dihydroxy aromatic such as bisphenol; polyvinylchlorides; polyamides such as polyhexamethylene adipamide and other such polyamides popularly known as nylon; modacrylic copolymers, such as styrene-acrylic acid copolymers; polysulfones such as those of the type characterized by diphenylene sulfone groups in the linear chain thereof are useful; halogenated polymers such as polyvinylidene fluoride, polyvinylfluoride, and polyfluorohalocarbons; polychloroethers and other such thermoplastic polyethers; acetal polymers such as poly formaldehydes; acrylic resins such as polyacrylonitrile polymethyl poly(vinyl alcohol), derivatives of polystyrene such as poly (sodium styrenesulfonate) and polyvinylbenzyltrimethyl-ammonium chloride), poly(hydroxyethyl methacrylate poly(isobutyl vinyl ether); and a large number of copolymers which can be formed by reacting various proportions of monomers from the aforesaid list of polymers are also useful for preparing rate controlling structures useful in the invention. In some embodiments, the microporous membrane includes polypropylene.

Without being bound to any particular theory, diffusion of an active agent through microporous polymeric materials such as microporous polypropylene can be difficult. The polymers are impermeable to the active drugs except at the pore channels, and even then the active agent cannot diffuse through the pores unless it does so in a vaporized state. Thus, if a microporous membrane is used as purchased in the fabrication of a transdermal delivery system, an excessive amount of time may be required for a delivery vehicle (i.e., drug matrix solvent composition) from a drug matrix layer to partition into the pores and then for the active agent to partition into the delivery vehicle within the pores. The resultant effect is that it can take a long time for the active agent to reach its intended target.

The release rate of an active agent through a microporous membrane can be greatly improved when the microporous membrane is pretreated with a suitable delivery vehicle or membrane solvent composition. Pretreated as used herein intend that the microporous membrane is exposed to a membrane solvent composition to fill pores within the microporous membrane prior to the microporous membrane's incorporation into a transdermal system. The pores of the microporous membrane are filled with or contain a membrane solvent composition prior to and at the time the microporous membrane is incorporated into the transdermal system. The release rate of an active agent through a microporous membrane depends on several variables such as the diffusivity and solubility of the active agent in the membrane solvent composition and the thickness and porosity of the microporous material. For flow of the active agent through the pores of the microporous membrane the concentration gradient, the thickness of the membrane, the viscosity of the active agent, the size of the active agent molecule relative to the pore size, the absolute value of the pore size, and the number of pores or percent voids (porosity) in the material are contributing factors governing solubility and diffusivity of an agent into and through the membrane.

In some embodiments, the microporous membrane comprises a plurality of pores. In some embodiments, the microporous membrane can have a porosity in the range of, but not limited to, about 30% to about 50%, about 35% to about 45%, or about 40% to about 42%. For example, the microporous membrane can have a porosity of, but not limited to, about 30%, 31%, 32%, 33%, 34%, 35%, 36%, 37%, 38%, 39%, 40%, 41%, 42%, 43%, 44%, 45%, 46%, 47%, 48%, 49%, or 50%.

In some embodiments, the microporous membrane can have an average pore size in the range of, but not limited to, about 0.001 μm to about 100 μm, about 1 μm to about 10 μm, about 0.010 μm to about 0.100 μm, or about 0.040 μm to about 0.050 μm. For example, the average pore size can be of, but not limited to, about 0.035 μm, 0.036 μm, 0.037 μm, 0.038 μm, 0.039 μm, 0.040 μm, 0.041 μm, 0.042 μm, 0.043 μm, 0.044 μm, 0.045 μm, 0.046 μm, 0.047 μm, 0.048 μm, 0.049 μm, or 0.050 μm. In some embodiments, the microporous membrane has an average pore size of about 0.043 μm.

The microporous membrane can be pretreated with the same or a different membrane solvent composition than the drug matrix solvent composition present in the drug matrix layer. In some embodiments, the microporous membrane is pretreated with a membrane solvent composition comprising a solvent, a surfactant, an emulsifier, a viscosity increasing agent, a stabilizer, a plasticizer, and/or combinations thereof. In some embodiments, the surfactant is a nonionic surfactant. In some embodiments, the microporous membrane is pretreated with a citrate ester. In some embodiments, the citrate ester is triethyl citrate. In some embodiments, the microporous membrane is pretreated with lauryl lactate. In some embodiments, the microporous membrane is pretreated with a sorbitan monoester. In some embodiments, the sorbitan monoester is sorbitan monolaurate (sorbitan laurate). In some embodiments, the membrane layer is pretreated with a membrane solvent composition comprising triethyl citrate, lauryl lactate, and sorbitan monolaurate. In some embodiments, the microporous membrane is pretreated with octyldodecanol.

In some embodiments, the microporous membrane has a plurality of pores that are filled with or that contain a membrane solvent composition that is different from the drug matrix solvent composition in the drug matrix layer in fluid communication with the microporous membrane. In some embodiments, the membrane solvent composition does not include (i.e., excludes) a solvent in which the salt form of the active agent is soluble. In some embodiments, the membrane solvent composition does not include (i.e., excludes) a hydrophilic solvent in which the salt form of the active agent is soluble. In some embodiments, the membrane solvent composition does not include (i.e., excludes) a polyol, including solvent polyols, such as polyethylene glycol, propylene glycol, glycerin (glycol), acetonitrile, 1-propanol, N,N-dimethylformamide and dimethyl sulfoxide.

Without being bound to any particular theory, the membrane solvent composition enables the base form of the active agent to be dissolved or suspended therein and move diffusionally into and through the microporous membrane.

The materials selected for the membrane solvent composition can be non-toxic and those in which the rate controlling microporous material has the required solubility. In some embodiments, the membrane solvent composition is not a solvent for the material from which the microporous membrane is manufactured. That is, the microporous membrane is chemically stable in the membrane solvent composition. The materials which are useful for impregnating, filling, or saturating the pores or micropores of the microporous membrane can be polar, semi-polar or non-polar. Materials for use in a membrane solvent composition in addition to those listed above include, but are not limited to, pharmaceutically acceptable alcohols containing 6 to 25 carbon atoms, such as hexanol, cyclohexanol, benzylalcohol, 1,2-butanediol, glycerol, and amyl alcohol, and octyldodecanol; hydrocarbons having 5 to 12 carbon atoms such as n-hexane, cyclohexane, and ethyl benzene; aldehydes and ketones having 4 to 10 carbon atoms such as heptyl aldehyde, cyclohexanone, and benzaldehyde; esters having 4 to 10 carbon atoms such as amyl acetate and benzyl propionate; ethereal oils such as oil of Eucalyptus, oil of rue, cumin oil, limonene, thyme, and 1-pinene; halogenated hydrocarbons having 2 to 8 carbon atoms such as n-hexyl chloride, n-hexyl bromide, and cyclohexyl chloride; or mixtures of any of the foregoing materials.

In some embodiments, the plurality of pores in the microporous membrane contain a membrane solvent composition comprised of one or more of triethyl citrate, sorbitan monolaurate, and lauryl lactate.

In some embodiments, the microporous membrane includes triethyl citrate. The triethyl citrate can be present in any suitable amount. For example, the membrane layer includes triethyl citrate in an amount of, but not limited to, about 50-99% (w/w), or about 55-95%, or about 55-90%, or about 55-85%, or about 55-80%, or about 60-75%, or about 61-74%, or about 62-73%, or about 63-72%, or about 64-71%, or about 65-70%, or about 66-69% (w/w). The membrane layer can also include triethyl citrate in an amount of, but not limited to, about 50% (w/w), or about 55, 60, 61, 62, 63, 64, 65, 66, 67, 68, 69, 70, 71, 72, 73, 74, 75, 80, 85, 90, or about 95% (w/w). In some embodiments, the membrane layer includes triethyl citrate in an amount of about 67% (w/w). In some embodiments, the membrane layer includes triethyl citrate in an amount of about 66.7% (w/w). In some embodiments, the membrane layer includes triethyl citrate in an amount of 66.7% (w/w). The weight percentages provided can represent the weight percentage of the triethyl citrate to the total weight of the membrane solvent composition.

In some embodiments, the microporous membrane includes lauryl lactate. The lauryl lactate can be present in any suitable amount. For example, the membrane layer can include lauryl lactate in an amount of, but not limited to, about 1-50% (w/w), or about 1-40%, or about 5-35%, or about 10-30%, or about 15-25%, or about 16-24%, or about 17-23%, or about 18-22%, or about 19-21% (w/w). The membrane layer can also include lauryl lactate in an amount of, but not limited to, about 5% (w/w), or about 10, 11, 12, 13, 14, 15, 16, 17, 18, 19, 20, 21, 22, 23, 24, 25, 26, 27, 28, 29, 30, 35, 40, 45, or about 50% (w/w). In some embodiments, the membrane layer includes lauryl lactate in an amount of about 20% (w/w). In some embodiments, the membrane layer includes lauryl lactate in an amount of about 20.0% (w/w). In some embodiments, the membrane layer includes lauryl lactate in an amount of 20.0% (w/w). The weight percentages provided can represent the weight percentage of lauryl lactate to the total weight of the membrane solvent composition.

In some embodiments, the microporous membrane includes sorbitan monolaurate. The sorbitan monolaurate can be present in any suitable amount. For example, the membrane layer can include sorbitan monolaurate in amount of, but not limited to, about 1-50% (w/w), or about 1-45%, or about 1-40%, or about 1-35%, or about 1-30%, or about 5-25%, or about 10-20%, or about 10-15%, or about 12-15% (w/w). The membrane layer can also include sorbitan monolaurate in an amount of, but not limited to, about 5% (w/w), or about 6, 7, 8, 9, 10, 11, 12, 13, 14, 15, 16, 17, 18, 19, 20, 21, 22, 23, 24, 25, 30, 35, 40, 45, or about 50% (w/w). In some embodiments, the membrane layer includes sorbitan monolaurate in an amount of about 13% (w/w). In some embodiments, the membrane layer includes sorbitan monolaurate in an amount of about 13.3% (w/w). In some embodiments, the membrane layer includes sorbitan monolaurate in an amount of 13.3% (w/w). The weight percentages provided can represent the weight percentage of sorbitan monolaurate to the total weight of the membrane solvent composition.

In some embodiments, the microporous membrane comprises polypropylene, and the plurality of pores in the microporous membrane comprises triethyl citrate, sorbitan monolaurate, and lauryl lactate.

In some embodiments, the membrane solvent composition comprises about 60% (w/w) to about 75% (w/w) triethyl citrate. In some embodiments, the membrane solvent composition includes triethyl citrate in an amount of, but not limited to, about 55% (w/w) to about 80% (w/w), about 60% (w/w) to about 70% (w/w), about 65% (w/w) to about 75% (w/w), or about 65% (w/w) to about 70% (w/w). In some embodiments, the membrane solvent composition includes sorbitan monolaurate in an amount of about 10% (w/w) to about 17% (w/w). In some embodiments, the membrane solvent composition includes sorbitan monolaurate in an amount of, but not limited to, about 8% (w/w) to about 25% (w/w), about 10% (w/w) to about 25% (w/w), about 8% (w/w) to about 17% (w/w), about 12% (w/w) to about 20% (w/w), about 10% (w/w) to about 15% (w/w), or about 12% (w/w) to about 14% (w/w). In some embodiments, the membrane solvent composition includes lauryl lactate in an amount of about 15% (w/w) to about 25% (w/w). In some embodiments, the membrane solvent composition includes lauryl lactate in an amount of, but not limited to, about 10% (w/w) to about 30% (w/w), about 15% (w/w) to about 30% (w/w), about 15% (w/w) to about 20% (w/w), about 10% (w/w) to about 25% (w/w), about 10% (w/w) to about 20% (w/w), about 17% (w/w) to about 23% (w/w), about 18% (w/w) to about 22% (w/w), or about 19% (w/w) to about 21% (w/w). In some embodiments, the membrane solvent composition can be formulated with the combination of triethyl citrate, lauryl lactate, and sorbitan monolaurate in any of the ranges recited above. In some embodiments, the membrane solvent composition comprises triethyl citrate in an amount of about 66.7% (w/w), lauryl lactate in an amount of about 20.0% (w/w), and sorbitan monolaurate in an amount of about 13.3% (w/w). In some embodiments, the membrane solvent composition comprises triethyl citrate in an amount of 66.7% (w/w), lauryl lactate in an amount of 20.0% (w/w), and sorbitan monolaurate in an amount of 13.3% (w/w). The weight percentages provided can represent the weight percentage of each component to the total weight of the membrane solvent composition.

The thickness of the microporous membrane can vary depending on the type of material and the desired characteristics of the microporous membrane (e.g., porosity, micropore size, time diffusion of the active agent through the membrane). In some embodiments, the microporous membrane has a thickness of between about 5 to about 200 μm. In some embodiments, the microporous membrane has a thickness of, but not limited to, about 10 to about 150 μm, about 10 to about 125 μm, about 10 to about 100 μm, about 10 to about 75 μm, about 10 to about 50 μm, about 5 to about 45 μm, about 5 to about 30 μm, about 10 to about 30 μm, about 15 to about 30 μm, or about 20 to about 30 μm. In some embodiments, the microporous membrane has a thickness of, but not limited to, about 22 to about 28 μm. In some embodiments, the microporous membrane has a thickness of about 24 to about 26 μm. In some embodiments, the microporous membrane, has a thickness of about 25 μm.

The microporous membrane can be pretreated in a variety of ways. In general, pretreating comprises contacting the microporous membrane with the membrane solvent composition in a sufficient manner and for a sufficient amount of time. In some embodiments, the pretreating of the microporous membrane comprises contacting the microporous membrane with the membrane solvent composition, allowing the microporous membrane to become saturated with the membrane solvent composition, and removing any excess membrane solvent composition from the saturated microporous membrane. In some embodiments, the microporous membrane is soaked in the membrane solvent composition. In some embodiments, the microporous membrane is immersed into a bath of the membrane solvent composition. In some embodiments, the membrane solvent composition is spread onto the microporous membrane until the microporous membrane is saturated and then the excess membrane solvent composition is removed.

The pretreatment of the microporous membrane with the membrane solvent composition can vary in degree. In some embodiments, a portion of the pores of the microporous membrane contain the membrane solvent composition therein. In some embodiments, about one third, about one half, about two thirds, or about three fourths of the pores will contain the membrane solvent composition. In some embodiments, all of the pores will contain the membrane solvent composition. In some embodiments, the portion of the pores containing membrane solvent composition will only be partially filled. In some embodiments, the membrane solvent composition will occupy about one fourth, about one third, about one half, about two thirds, or about three fourths of the space within the occupied pores. In some embodiments, all of the pores of the microporous membrane will be completely filled with the membrane solvent composition and the microporous membrane will thus be saturated with the membrane solvent composition.

### Contact Adhesive Layer

The transdermal delivery system of the present invention includes a contact adhesive layer. The contact adhesive layer can include a variety of components, such as a polymer or copolymer.

In some embodiments, the contact adhesive layer comprises one or more biocompatible polymers selected from one or more of polyisobutylene (PIB), a silicone polymer, acrylate copolymers, butyl rubber, polybutylene, styrene-isoprene-styrene block copolymers, styrene-butadiene-styrene block copolymers, ethylene-vinyl acetate (EVA), mixtures and copolymers thereof. In some embodiments, the biocompatible polymer is polyisobutylene.

A contact adhesive layer as described herein and hereinabove is contemplated for use in a transdermal delivery system, where the system additionally comprises an adhesive component. The contact adhesive layer can include the adhesive component in an amount of, but not limited to, about 50-90% (w/w) of adhesive polymer or copolymer, or between about 55-90% (w/w), or between about 60-90% (w/w), between about 65-90% (w/w), between about 70-90% (w/w), between about 75-90% (w/w), or between about 80-90% (w/w). In some embodiments, the contact adhesive layer includes a copolymer of acrylate/vinyl acetate. In some embodiments, the contact adhesive layer includes a polyvinylpyrrolidone, such as a crosslinked polyvinylpyrrolidone.

The adhesive polymer component of the contact adhesive layer can be any suitable adhesive materials, such as pressure sensitive adhesive polymers. Polyacrylate pressure sensitive adhesive polymers are an example, and typically comprise a polyacrylate that is a polymer or a copolymer of a monomer or monomers selected from acrylic acid esters and methacrylic acid esters. Other monomers, such as acrylic acid and vinyl acetate, may be present. In some embodiments, the acrylic polymer is based on acrylic esters such as 2-ethylhexyl acrylate (2-EHA) and ethyl acrylate. In some embodiments, the polyacrylate polymer is a polymer or a copolymer of a monomer or monomers selected from acrylic acid and vinyl acetate. In some embodiments, the acrylic polymer adhesive has pendent carboxyl (—COOH) or hydroxyl (—OH) functional groups. In some embodiments, the acrylic polymer adhesive comprises at least one of polyacrylate, polymethacrylate, derivatives thereof, and co-polymers thereof. In some embodiments, the acrylic adhesive is comprised of an acrylate copolymer comprising acrylic ester monomers, acrylic acid, and/or vinyl acetate monomers. A copolymer of acrylic acid and vinyl acetate is one example. Acrylate copolymers are sold under the trade-name DURO-TAK® and include, but are not limited to, DURO-TAK 87-2287, 387-2516, 387-2051, and 387-2074. In some embodiments, the acrylate polymer comprises DURO-TAK 82-2287.

In some embodiments, the contact adhesive layer comprises at least about 25-80% (w/w) of adhesive polymers relative to the weight of the contact adhesive layer (inclusive of sub-ranges). In some embodiments, the contact adhesive layer includes an adhesive polymer or copolymer or mixture of polymers and/or copolymers in an amount of, but not limited to, about 35-80%, 30-75%, about 40-75%, about 50-75%, about 60-75%, about 25-70%, about 30-70%, about 40-70%, about 50-70%, about 60-70%, about 25-60%, about 30-60%, about 40-60%, about 50-60%, about 25-50%, about 30-50%, about 40-50%, about 25-40%, about 30-40%, or about 25-30% (w/w). The contact adhesive layer can include one or more adhesive polymers or copolymers. In some embodiments, the contact adhesive layer includes about 5-75% of an individual polymer relative to the total weight of the polymers in the contact adhesive layer. In some embodiments, the contact adhesive layer includes an individual polymer in an amount of, but not limited to, about 5-10%, 5-15%, 5-20%, 5-25%, 5-30%, 5-40%, 5-50%, 5-60%, 5-70%, 5-75%, 10-15%, 10-20%, 10-20%, 10-25%, 10-30%, 10-40%, 10-50%, 10-60%, 10-70%, 10-75%, 15-20%, 15-25%, 15-30%, 15-40%, 15-50%, 15-60%, 15-70%, 15-75%, 20-25%, 20-30%, 20-40%, 20-50%, 20-60%, 20-70%, 20-75%, 25-30%, 25-40%, 25-50%, 25-60%, 25-70%, 25-75%, 30-40%, 30-50%, 30-60%, 30-70%, 30-75%, 40-50%, 40-60%, 40-70%, 40-75%, 50-60%, 50-70%, 50-75%, 60-70%, 60-75%, or 70-75% (w/w). In some embodiments, the contact adhesive layer includes the acrylate polymer in an amount of from 50-75% (w/w). In some embodiments, the contact adhesive layer includes the acrylate polymer in an amount of from 60-70% (w/w). In some embodiments, the contact adhesive layer includes the acrylate polymer in an amount of from 63-65% (w/w). In some embodiments, the contact adhesive layer includes the acrylate polymer in an amount of about 64% (w/w). In some embodiments, the contact adhesive layer includes the acrylate polymer in an amount of about 64.6% (w/w). In some embodiments, the contact adhesive layer includes the acrylate polymer in an amount of 64.6% (w/w). The weight percentages provided can represent the weight percentage of the acrylate polymer to the total weight of the contact adhesive layer.

In some embodiments, the contact adhesive layer comprises a copolymer of acrylic acid and vinyl acetate. In some embodiments, the contact adhesive layer includes Duro-Tak 87-2287 in an amount of about 64.6% (w/w). In some embodiments, the contact adhesive layer includes Duro-Tak 87-2287 in an amount of 64.6% (w/w). The weight percentages provided can represent the weight percentage of the Duro-Tak 87-2287 to the total weight of the contact adhesive layer.

The contact adhesive layer can also include one or more solvents. The contact adhesive layer also comprises a contact adhesive solvent composition. In some embodiments, the contact adhesive solvent composition includes one, two, three or four solvents. In some embodiments, the contact adhesive solvent composition comprises triethyl citrate; and in other embodiments, one or both of lauryl lactate and sorbitan monolaurate are additionally present. In some embodiments, the contact adhesive solvent composition is comprised of, consists essentially of, or consists of triethyl citrate, sorbitan monolaurate, and lauryl lactate.

In some embodiments, the contact adhesive layer can include one or more of methyl laurate, propylene glycol monolaurate, glycerol monolaurate, glycerol monooleate, lauryl lactate, myristyl lactate, and dodecyl acetate. Additional contact adhesive solvent compositions are described in U.S. Pat. No. 8,874,879, which is incorporated herein by reference.

In some embodiments, the contact adhesive layer includes the contact adhesive solvent composition in an amount of about 5-50% (w/w) of contact adhesive solvent composition relative to the weight of the contact adhesive layer (inclusive of sub-ranges). In some embodiments, the contact adhesive layer includes the contact adhesive solvent composition in an amount of, but not limited to, about 5-45%, 5-40%, 5-35%, 5-30%, 5-25%, 10-20, 11-19, 12-18, 13-17, or 14-16% (w/w). Alternatively, the contact adhesive layer includes the contact adhesive solvent composition in an amount of, but not limited to, about 10% (w/w), or about 11, 12, 13, 14, 15, 16, 17, 18, 19, 20, 21, 22, 23, 24, or about 25% (w/w). In some embodiments, the contact adhesive layer includes the contact adhesive solvent composition in an amount of about 15% (w/w). In some embodiments, the contact adhesive layer includes the contact adhesive solvent composition in an amount of about 15.6% (w/w). In some embodiments, the contact adhesive layer includes the contact adhesive solvent composition in an amount of 15.6% (w/w). The weight percentages provided can represent the weight percentage of the contact adhesive solvent composition to the total weight of the contact adhesive layer.

In some embodiments, the contact adhesive solvent composition of the contact adhesive layer includes triethyl citrate. The triethyl citrate can be present in in any suitable amount in the contact adhesive layer. For example, the contact adhesive solvent composition of the contact adhesive layer can include triethyl citrate in an amount of, but not limited to, about 1-20% (w/w), or about 2-19%, or about 3-18%, or about 4-17%, or about 5-16%, or about 5-15%, or about 6-15%, or about 7-15%, or about 8-14%, or about 9-13%, or about 9-11% (w/w). Alternatively, the contact adhesive layer includes triethyl citrate in an amount of, but not limited to, about 5% (w/w), or about 6, 7, 8, 9, 10, 11, 12, 13, 14, or about 15% (w/w). In some embodiments, the contact adhesive layer includes triethyl citrate in an amount of about 10% (w/w). In some embodiments, the contact adhesive layer includes triethyl citrate in an amount of about 10.5% (w/w). In some embodiments, the contact adhesive layer includes triethyl citrate in an amount of 10.5% (w/w). The weight percentages provided can represent the weight percentage of the triethyl citrate to the total weight of the contact adhesive layer.

In some embodiments, the contact adhesive solvent composition of the contact adhesive layer includes lauryl lactate. The lauryl lactate can be present in any suitable amount in the contact adhesive layer. For example, the contact adhesive solvent composition of the contact adhesive layer can include lauryl lactate in an amount of, but not limited to, about 0.1-10% (w/w), or about 0.5-10%, or about 1-10%, or about 1-5%, or about 2-4% (w/w). Alternatively, the contact adhesive layer includes lauryl lactate in an amount of, but not limited to, about 1% (w/w), or about 1.5, 2.0, 2.5, 2.6, 2.7, 2.8, 2.9, 3.0, 3.1, 3.2, 3.3, 3.4, 3.5, 3.6, 3.7, 3.8, 3.9, 4.0, 4.5, or about 5.0% (w/w). In some embodiments, the contact adhesive layer includes lauryl lactate in an amount of about 3% (w/w). In some embodiments, the contact adhesive layer includes lauryl lactate in an amount of about 3.1% (w/w). In some embodiments, the contact adhesive layer includes lauryl lactate in an amount of 3.1% (w/w). The weight percentages provided can represent the weight percentage of the lauryl lactate to the total weight of the contact adhesive layer.

In some embodiments, the contact adhesive solvent composition of the contact adhesive layer includes sorbitan monolaurate. The sorbitan monolaurate can be present in any suitable amount in the contact adhesive layer. For example, the contact adhesive layer can include sorbitan monolaurate in an amount of, but not limited to, about 0.1-10% (w/w), or about 0.1-5%, or about 0.5-5%, or about 1-5%, or about 1-3% (w/w). Alternatively, the contact adhesive layer can include sorbitan monolaurate in an amount of, but not limited to, about 1% (w/w), or about 1.1, 1.2, 1.3, 1.4, 1.5, 1.6, 1.7, 1.8, 1.9, 2.0, 2.1, 2.2, 2.3, 2.4, or about 2.5% (w/w). In some embodiments, the contact adhesive layer includes sorbitan monolaurate in an amount of about 2% (w/w). In some embodiments, the contact adhesive layer includes sorbitan monolaurate in an amount of about 2.0% (w/w). In some embodiments, the contact adhesive layer includes sorbitan monolaurate in an amount of 2.0% (w/w). The weight percentages provided can represent the weight percentage of the sorbitan monolaurate to the total weight of the contact adhesive layer.

In some embodiments, the contact adhesive layer further comprises one or more solvents of triethyl citrate, sorbitan monolaurate, or lauryl lactate.

In some embodiments, the contact adhesive layer is manufactured from an adhesive formulation that does not comprise donepezil HCl or donepezil free base. Without being bound by any particular theory, while the contact adhesive layer is not manufactured with donepezil HCl or donepezil free base, the donepezil free base can migrate from the drug matrix layer into the contact adhesive layer following preparation of the transdermal delivery system and prior to administration of the transdermal delivery system to the subject.

In some embodiments, the contact adhesive layer includes donepezil free base. In some embodiments, the contact adhesive layer includes donepezil free base prior to administration of the transdermal delivery system to the subject. The donepezil free base can be present in any suitable amount in the contact adhesive layer. For example, the contact adhesive layer can include donepezil free base in an amount of, but not limited to, about 0.1-10% (w/w), or about 0.1-5%, or about 0.5-5%, or about 1-5%, or about 1-6%, or about 2-5%, or about 3-5%, or about 4-5%, or about 1-4%, or about 1-3%, or about 1-2%, or about 2-4%, or about 2-3%, or about 3-4% (w/w). Alternatively, the contact adhesive layer can include donepezil free base in an amount of, but not limited to, about 1% (w/w), or about 1.1, 1.2, 1.3, 1.4, 1.5, 1.6, 1.7, 1.8, 1.9, 2.0, 2.1, 2.2, 2.3, 2.4, or about 2.5% (w/w). In some embodiments, the contact adhesive layer includes donepezil free base in an amount of at least 0.1% (w/w). In some embodiments, the contact adhesive layer includes donepezil free base in an amount of at least 1% (w/w). In some embodiments, the contact adhesive layer includes donepezil free base in an amount of about 2% (w/w). In some embodiments, the contact adhesive layer includes donepezil free base in an amount of about 2% (w/w). In some embodiments, the contact adhesive layer includes donepezil free base in an amount of about 2.0% (w/w). In some embodiments, the contact adhesive layer includes donepezil free base in an amount of from 1-5% (w/w). In some embodiments, the contact adhesive layer includes donepezil free base in an amount of from 2-4% (w/w). In some embodiments, the contact adhesive layer includes donepezil free base in an amount of from 2-3% (w/w). In some embodiments, the contact adhesive layer includes donepezil free base in an amount of 2.0% (w/w). Without being bound to any particular theory, the donepezil free base present in the contact adhesive layer is administered to the subject following application of the transdermal delivery system of the present invention to the subject's skin. The weight percentages provided can represent the weight percentage of the donepezil free base to the total weight of the contact adhesive layer.

The contact adhesive layer can also comprise a contact adhesive solvent composition. In some embodiments, the contact adhesive layer comprises a contact adhesive solvent of one or more of a citric ester, a surfactant and/or an α-hydroxy acid. In some embodiments, the contact adhesive layer comprises a contact adhesive solvent composition of one or more of triethyl citrate, sorbitan monolaurate, and/or lauryl lactate. In some embodiments, the contact adhesive layer as manufactured does not include a pharmaceutically active agent intended for systemic delivery, for example, the ingredients combined to form the contact adhesive layer and/or the contact adhesive solvent composition do not include a base form or a salt form of a drug, such as donepezil free base or a donepezil salt. During use, after the contact adhesive layer is applied to the skin of a subject, the base form of the active agent that is in the drug matrix layer partitions into the drug matrix solvent composition in the drug matrix layer, then partitions and moves into the membrane layer solvent composition in the microporous membrane, and then partitions and moves into the contact adhesive solvent composition for delivery to the skin of the subject.

In some embodiments, the contact adhesive layer optionally comprises highly dispersive silica, e.g., hydrophobic colloidal silica that can effectively adsorb hydrophobic drugs and other hydrophobic ingredients. By using hydrophobic colloidal silica at a certain percentage as an excipient (from about 3% to about 20%, preferably from about 5% to about 10% in the formulation), the diffusion of the active ingredient through the matrix can be controlled during storage. Examples of the dispersive silica for use in the compositions include, but are not limited to, the high purity amorphous anhydrous colloidal silicon dioxide for use in pharmaceutical products sold under the name AEROSIL, e.g., AEROSIL®90, AEROSIL®130, AEROSIL®150, AEROSIL®200, AEROSIL®300, AEROSIL®380, AEROSIL®OX50, AEROSIL®TT600, AEROSIL®MOX80, AEROSIL®COK84, AEROSIL®R202, AEROSIL®R805, AEROSIL®R812, AEROSIL®812S, AEROSIL®R972, and/or AEROSIL® R974 or any other highly disperse silica, especially AEROSIL®200 and/or AEROSIL®R972 can be used as highly disperse silica.

In some embodiments, the contact adhesive layer comprises highly dispersive silica at least about 40% by weight relative to the weight of the entire adhesive layer, including, at least about 1% by weight relative to the weight of the adhesive layer, including, at least about 3%, e.g., about 4%, about 5%, about 6%, about 7%, about 8%, about 9%, about 10%, about 11%, about 12%, about 13%, about 14%, about 15%, about 16%, about 17%, about 18%, about 19%, about 20%, or greater % by weight, wherein all values are relative to the weight of the entire adhesive layer.

The contact adhesive layer may further include one or more matrix modifiers. Without wishing to be bound by theory, it is believed that the matrix modifier facilitates homogenization of the adhesive matrix. Sorption of hydrophilic moieties is a possible mechanism for this process. Thus, known matrix modifiers which are to some degree water-sorbent may be used. For example, possible matrix modifiers include colloidal silicone dioxide, fumed silica, cross-linked polyvinylpyrrolidone (PVP), soluble PVP, cellulose derivatives (e.g. hydroxypropyl cellulose (HPC), hydroxyethylcellulose (HEC)), polyacrylamide, polyacrylic acid, a polyacrylic acid salt, or a clay such as kaolin or bentonite. An exemplary commercial fumed silica product is Cab-O-Sil (Cabot Corporation, Boston, Mass.). The hydrophilic mixtures described in U.S. Published Patent Application No. 2003/0170308 may also be employed, for example mixtures of PVP and PEG or of PVP, PEG, and a water-swellable polymer such as EUDRAGIT® L100-55. In some embodiments, the matrix modifier is individually included in an amount between about 1-25%, about 2-25%, about 5-25%, about 5-7%, about 7-20%, or about 7-25% relative to the weight of the adhesive matrix (inclusive of sub-ranges). In some embodiments, the matrix modifier does not include ethylcellulose.

The contact adhesive layer may also comprise a copolymer such as a polyvinylpyrrolidone/vinyl acetate copolymer, an acrylate/vinyl acetate copolymer, or a vinyl acetate/ethylene acetate copolymer. In some embodiments, the copolymer is a vinyl acetate/N-vinylpyrrolidone copolymer such as the copolymer sold as Plasdone™ S630 (Ashland). In some embodiments, the polyvinylpyrrolidone-vinyl acetate copolymer is a linear random copolymer of n-vinyl-2-pyrrolidone and vinyl acetate. In some embodiments, the copolymer is a 60:40 copolymer of n-vinyl-2-pyrrolidone and vinyl acetate.

The contact adhesive layer may also comprise a polyvinylpyrrolidone (PVP). PVP is a water-soluble polymer comprised of the N-vinylpyrrolidone monomer, and is available in various forms, including cross-linked and non-crosslinked. In some of the working examples herein, a cross-linked PVP is included in the contact adhesive layer. In some embodiments, the cross-linked PVP is Crospovidone. In some embodiments, the contact adhesive layer further comprises Crospovidone.

The Crospovidone can be present in the contact adhesive layer in any suitable amount. For example, the Crospovidone be present in the contact adhesive layer in an amount of, but not limited to, from 1-50% (w/w), or 5-25%, or 10-20%, or 11-19%, or 12-18%, or 13-17%, or 14-16% (w/w). Alternatively, the contact adhesive layer includes Crospovidone in an amount of, but not limited to, about 19.0% (w/w), or about 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, or 21.0% (w/w). In some embodiments, the contact adhesive layer includes Crospovidone in an amount of about 20% (w/w). In some embodiments, the contact adhesive layer includes Crospovidone in an amount of about 19.9% (w/w). In some embodiments, the contact adhesive layer includes Crospovidone in an amount of 19.9% (w/w). The weight percentages provided can represent the weight percentage of the Crospovidone to the total weight of the contact adhesive layer.

In some embodiments, the contact adhesive layer includes acrylate-vinyl acetate copolymer in an amount of about 64.6% (w/w), triethyl citrate in an amount of 10.5% (w/w), lauryl lactate in an amount of about 3.1% (w/w), sorbitan monolaurate in an amount of about 2.0% (w/w), and Crospovidone in an amount of about 19.9% (w/w). In some embodiments, the contact adhesive layer includes acrylate-vinyl acetate copolymer in an amount of 64.6% (w/w), triethyl citrate in an amount of 10.5% (w/w), lauryl lactate in an amount of 3.1% (w/w), sorbitan monolaurate in an amount of 2.0% (w/w), and Crospovidone in an amount of 19.9% (w/w). The weight percentages provided can represent the weight percentage of each component to the total weight of the contact adhesive layer.

In some embodiments, the present invention provides a transdermal delivery system, comprising:
(1) a backing layer;
(2) a separating layer having a top surface and a bottom surface such that the top surface is in contact with the backing layer;
(3) a drug matrix layer comprising donepezil HCl, donepezil free base, and sodium bicarbonate, wherein the drug matrix layer has a top surface and a bottom surface such that the top surface is in contact with the bottom surface of the separating layer, and wherein the donepezil free base is present in an amount of at least 10% (w/w) of the total amount of donepezil free base and donepezil HCl;
(4) a membrane layer comprising a microporous membrane, wherein the membrane layer has a top surface and a bottom surface such that the top surface is in contact with the bottom surface of the drug matrix layer; and
(5) a contact adhesive layer having a top surface and a bottom surface such that the top surface is in contact with the bottom surface of the membrane layer, wherein the contact adhesive layer comprises donepezil free base in an amount of from 0.1 to 10% (w/w) of the total weight of the contact adhesive layer.

### Release Liner

The transdermal delivery system of the present invention can also include a release liner. The release liner can be attached to any other layer of the transdermal delivery system. In some embodiments, the transdermal delivery system includes a release liner at least partially in contact at least with the contact adhesive layer to protect the contact adhesive layer prior to application. In some embodiments, the transdermal delivery system also includes a release layer in contact with the bottom surface of the contact adhesive layer.

The release liner is typically a disposable layer that is removed prior to application of the device to the treatment site. In some embodiments, the release liner preferably does not absorb components of the contact adhesive layer, including the active agent. In some embodiments, the release liner is impermeable to components of the contact adhesive layer (including the active agent) and prevents release of components of the contact adhesive layer through the release liner. In some embodiments, the release liner is formed of one or more of a film, non-woven fabric, woven fabric, laminate, and combinations thereof. In some embodiments, the release liner is a silicone-coated polymer film or paper. In some non-limiting embodiments, the release liner is a silicone-coated polyethylene terephthalate (PET) film, a fluorocarbon film, or a fluorocarbon coated PET film.

In some embodiments, the release layer comprises a silicone coated material, a fluorocarbon coated material, or a fluorosilicone coated material. In some embodiments, the release layer comprises a silicone coated material.

The transdermal delivery system of the present invention can have a variety of configurations, as shown in FIG. 1C. FIG. 1C shows the transdermal delivery system 10 having the backing layer 20 and adhesive overlay layer 21, the separating layer 30 having the top surface 31 and the bottom surface 32, wherein the top surface 31 includes the ethyl vinyl acetate coating that has been treated with the high-energy discharge, the drug matrix layer 40 having the top surface 41 and the bottom surface 42, the membrane layer 50 having the top surface 51 and the bottom surface 52, the contact adhesive layer 60 having the top surface 61 and the bottom surface 62, and the release liner 70.

### VI. Methods of Treatment

A method for delivering a therapeutic agent transdermally to a subject is provided. In some embodiments, the present invention provides a method for transdermally administering donepezil free base, comprising: (i) removing a release liner from the transdermal delivery system of the present invention; and (ii) adhering the transdermal delivery system to the skin of a subject for a period up to about 10 days to deliver the donepezil free base to said subject.

In some embodiments, the method comprises treatment of one or more central nervous system (CNS) disorders using delivery systems described herein. Examples of CNS disorders include, but are not limited to, dementia (e.g., Alzheimer's disease, Parkinson's disease, Picks disease, fronto-temporal dementia, vascular dementia, normal pressure hydrocephalus, Huntington's disease (HD), and mild cognitive impairment (MCI)), neuro-related conditions, dementia-related conditions, such as epilepsy, seizure disorders, acute pain, chronic pain, chronic neuropathic pain may be treated using the systems and methods described herein. Epileptic conditions include complex partial, simple partial, partials with secondary generalization, generalized-including absence, grand mal (tonic clonic), tonic, atonic, myoclonic, neonatal, and infantile spasms. Additional specific epilepsy syndromes are juvenile myoclonic epilepsy, Lennox-Gastaut, mesial temporal lobe epilepsy, nocturnal frontal lobe epilepsy, progressive epilepsy with mental retardation, and progressive myoclonic epilepsy. The systems and methods described herein are also useful for the treatment and prevention of pain caused by disorders including cerebrovascular disease, motor neuron diseases (e.g. amyotrophic lateral sclerosis (ALS), Spinal motor atrophies, Tay-Sach's, Sandoff disease, familial spastic paraplegia), neurodegenerative diseases (e.g., familial Alzheimer's disease, prion-related diseases, cerebellar ataxia, Friedrich's ataxia, SCA, Wilson's disease, retinitis pigmentosa (RP), ALS, Adrenoleukodystrophy, Menke's Sx, cerebral autosomal dominant arteriopathy with subcortical infarcts (CADASIL); spinal muscular atrophy, familial ALS, muscular dystrophies, Charcot Marie Tooth diseases, neurofibromatosis, von-Hippel Lindau, Fragile X, spastic paraplesia, psychiatric disorders (e.g., panic syndrome, general anxiety disorder, phobic syndromes of all types, mania, manic depressive illness, hypomania, unipolar depression, depression, stress disorders, posttraumatic stress disorder (PTSD), somatoform disorders, personality disorders, psychosis, and schizophrenia), and drug dependence (e.g., alcohol, psychostimulants (e.g., crack, cocaine, speed, meth), opioids, and nicotine), Tuberous sclerosis, and Wardenburg syndrome), strokes (e.g., thrombotic, embolic, thromboembolic, hemorrhagic, venoconstrictive, and venous), movement disorders (e.g., Parkinson's disorder (PD), dystonias, benign essential tremor, tardive dystonia, tardive dyskinesia, and Tourette's syndrome), ataxic syndromes, disorders of the sympathetic nervous system (e.g., Shy Drager, Olivopontoicerebellar degeneration, striatonigral degeneration, Parkinson's disease (PD), Huntington's disease (HD), Guillain-Barre, causalgia, complex regional pain syndrome types I and II, diabetic neuropathy, and alcoholic neuropathy), Cranial nerve disorders (e.g., Trigeminal neuropathy, trigeminal neuralgia, Menier's syndrome, glossopharangela neuralgia, dysphagia, dysphonia, and cranial nerve palsies), myelopethies, traumatic brain and spinal cord injury, radiation brain injury, multiple sclerosis, Post-meningitis syndrome, prion diseases, myelities, radiculitis, neuropathies (e.g., Guillain-Barre, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy), axonic brain damage, encephalopathies, and chronic fatigue syndrome. The systems and methods described herein are also useful for the treatment multiple sclerosis, in particular relapsing-remitting multiple sclerosis, and prevention of relapses in multiple sclerosis and/or in relapsing-remitting multiple sclerosis. All of the above disorders may be treated with the systems and methods described herein.

In some embodiments, compositions and devices comprising donepezil are useful for treating, delaying progression, delaying onset, slowing progression, preventing, providing remission, and improvement in symptoms of cognitive disorders or disease are provided herein. In some embodiments, compositions and devices comprising donepezil are provided for maintaining mental function including, but not limited to a least one of maintaining thinking, memory, speaking skills as well as managing or moderating one or more behavioral symptoms of a cognitive disorder or disease. In some embodiments, the cognitive disorder is Alzheimer's disease. In some embodiments, the cognitive disorder is Alzheimer's type dementia. In some embodiments, compositions and devices comprising donepezil are provided for use in treating, etc. mild, moderate, or severe Alzheimer's disease.

In some embodiments, the therapeutic embodiments are carried out by contacting a tissue of a subject, e.g., skin tissue, with the transdermal delivery systems provided herein.

In some embodiments, the therapeutic embodiments are carried out by transdermally administering the active agent to a subject, e.g., a subject suffering from a CNS disorder such as Alzheimer's disease and/or dementia. The term “administering” means applying as a remedy, such as by the placement of an active agent in a manner in which such drug would be received, e.g., transdermally, and be effective in carrying out its intended purpose.

Treatment of a subject with the systems may be monitored using methods known in the art. See, e.g., Forchetti et al, “Treating Patients with Moderate to Severe Alzheimer's Disease: Implications of Recent Pharmacologic Studies.” Prim Care Companion J Clin Psychiatry, 7 (4): 155-161, 2005 (PMID: 16163398). The efficacy of treatment using the system is preferably evaluated by examining the subject's symptoms in a quantitative way, e.g., by noting a decrease in the frequency of adverse symptoms, behaviors, or attacks, or an increase in the time for sustained worsening of symptoms. In a successful treatment, the subject's status will have improved (i.e., frequency of relapses will have decreased, or the time to sustained progression will have increased).

Based on the exemplary transdermal delivery systems (also referred to as transdermal devices or devices) described herein, a method for treating a suitable condition with an active agent is provided. In some embodiments, devices comprising the active agent are useful for treating, delaying progression, delaying onset, slowing progression, preventing, providing remission, and improvement in symptoms of cognitive disorders or disease and of multiple sclerosis are provided herein. In some embodiments, devices comprising the active agent are provided for maintaining mental function including, but not limited to a least one of maintaining thinking, memory, speaking skills as well as managing or moderating one or more behavioral symptoms of a cognitive disorder or disease. In some embodiments, the cognitive disorder is Alzheimer's disease. In some embodiments, the cognitive disorder is Alzheimer's type dementia. In some embodiments, devices comprising donepezil are provided for use in treating, etc. mild, moderate, or severe Alzheimer's disease. In other embodiments, devices comprising fingolimod are provided for use in treating multiple sclerosis, preventing and/or reducing frequency of relapses of multiple sclerosis, in particular of relapsing-remitting multiple sclerosis.

In some embodiments, the methods relate to therapy of CNS disorders or of autoimmune disorders in a subject in need thereof by contacting a tissue of the subject with one or more transdermal delivery systems. The terms “transdermal” and “topical” are used herein in the broadest sense to refer to administration of an active agent, e.g., memantine or donepezil or fingolimod, to the skin surface or mucosal membrane of an animal, including humans, so that the drug passes through the body surface, e.g., skin, and into the individual's blood stream.

Alzheimer's disease is the most common cause of senile dementia and is characterized by cognitive deficits related to degeneration of cholinergic neurons. Alzheimer's affects 6-8% of people over the age of 65 and nearly 30% of people over the age of 85 (Sozio et al., Neuropsychiatric Disease and Treatment, 2012, 8:361-368), involving the loss of cognitive functioning and behavioral abilities. The causes of Alzheimer's disease are not yet fully understood. As Alzheimer's disease is associated with reduced levels of several cerebral neurotransmitters including acetylcholine (Ach), current treatment includes administering cholinesterase inhibitors. Cholinesterase inhibitors reduce the hydrolysis of acetylcholine in the synaptic cleft by inhibiting cholinesterase and/or butyrylcholinesterase, which increases acetylcholine levels resulting in improved neurotransmission (Id).

In some embodiments, the present invention provides a method of treating Alzheimer's disease, comprising applying to skin of a subject a transdermal delivery system of the present invention to deliver donepezil free base to the subject, thereby treating Alzheimer's disease.

In some embodiments, the present invention provides a method for transdermal delivery of donepezil free base, comprising: securing, or instructing to secure, a transdermal delivery system of the present invention to the skin of a subject to deliver the base form of the active agent from the system to the skin, wherein (i) the time to reach steady state flux is at least about 20% faster compared to a system with no membrane solvent composition in the pores of the microporous membrane, (ii) the system achieves its steady state equilibrium flux at least 20% faster compared to a system with no membrane solvent composition in the pores of the microporous membrane; and/or (iii) the active agent diffuses from the system to the skin at least 20% faster compared to a system with no membrane solvent composition in the pores of the microporous membrane.

The transdermal devices described herein may be designed for long term use and/or continuous administration of the active agent. The FDA has approved daily oral doses of donepezil of 5 mg, 10 mg, and 23 mg. It will be appreciated that the total dose of the active agent per transdermal device will be determined by the size of the device and the loading of the active agent within the adhesive matrix. In an embodiment, the active agent is donepezil in free base form. Lower drug loading of donepezil free base may be effective as compared to the salt form (e.g. donepezil hydrochloride). The ability to include lower drug loading to achieve efficacy results in a lower profile for the device (thinner) and/or smaller size, both of which are desirable to reduce discomfort. In some embodiments, the application period for the transdermal device is between about 1-10 days, 1-7 days, 1-5 days, 1-2 days, 3-10 days, 3-7 days, 3-5 days, 5-10 days, and 5-7 days inclusive. In some embodiments, the active agent is released from the adhesive matrix as a continuous and/or sustained release over the application period.

A method for delivering donepezil free base transdermally to a subject is provided. In the method a transdermal delivery system is applied to the skin, and upon application of the transdermal delivery system to the skin of a subject, transdermal delivery of the donepezil free base occurs, to provide a systemic blood concentration of the agent (or a metabolite) that at steady state is bioequivalent to administration of the therapeutic agent orally. As discussed below, bioequivalency is established by (a) a 90% confidence interval of the relative mean Cmax and AUC of the therapeutic agent administered from the transdermal delivery system and via oral delivery are between 0.80 and 1.25 or between 0.70-1.43, or (b) a 90% confidence interval of the geometric mean ratios for AUC and Cmax of the therapeutic agent administered from the transdermal delivery system and via oral delivery are between 0.80 and 1.25 or between 0.70-1.43.

Standard PK parameters routinely used to assess the behavior of a dosage form in vivo (in other words when administered to an animal or human subject) include Cmax (peak concentration of drug in blood plasma), Tmax (the time at which peak drug concentration is achieved) and AUC (the area under the plasma concentration vs time curve). Methods for determining and assessing these parameters are well known in the art. The desirable pharmacokinetic profile of the transdermal delivery systems described herein comprise but are not limited to: (1) a Cmax for transdermally delivered form of the donepezil when assayed in the plasma of a mammalian subject following administration, that is bioequivalent to the Cmax or an orally delivered or an intravenously delivered form of the drug, administered at the same dosage; and/or (2) an AUC for transdermally delivered form of donepezil when assayed in the plasma of a mammalian subject following administration, that is preferably bioequivalent to the AUC for an orally delivered or an intravenously delivered form of the drug, administered at the same dosage; and/or (3) a Tmax for transdermally delivered form of donepezil when assayed in the plasma of a mammalian subject following administration, that is within about 80-125% of the Tmax for an orally delivered or an intravenously delivered form of the drug, administered at the same dosage. Preferably the transdermal delivery system exhibits a PK profile having a combination of two or more of the features (1), (2) and (3) in the preceding sentence. Preferably the transdermal delivery system exhibits a PK profile having one or both of the features (1) and (2).

In the field of pharmaceutical development the term “bioequivalence” will be readily understood and appreciated by the person skilled in the art. Various regulatory authorities have strict criteria and tests for assessing whether or not two drug products are bioequivalent. These criteria and tests are commonly used throughout the pharmaceutical industry and the assessment of bioequivalence is recognized as a standard form of activity in drug development programs where the characteristics and performance of one product are being compared to those of another product. Indeed in seeking approval to market certain types of products (e.g. those evaluated under the FDA's “Abbreviated New Drug Application” procedure), it is a requirement that the follow-on product be shown to be bioequivalent to a reference product.

In some embodiments, the method encompasses providing and/or administering a transdermal delivery system comprising donepezil free base to a subject in a fasted state is bioequivalent to administration of the agent (in base or salt form) orally or intravenously to a subject also in a fasted state, in particular as defined by Cmax and AUC guidelines given by the U.S. Food and Drug Administration and the corresponding European regulatory agency (EMEA). In some embodiments, the method encompasses providing and/or administering a transdermal delivery system comprising donepezil free base to a subject in a fasted state is bioequivalent to administration of the agent (in base or salt form) orally or intravenously to a subject also in a non-fasted or fed state. Under U.S. FDA and Europe's EMEA guidelines, two products or methods are bioequivalent if the 90% Confidence Intervals (CI) for AUC and Cmax are between 0.80 to 1.25 (Tmax measurements are not relevant to bioequivalence for regulatory purposes). Europe's EMEA previously used a different standard, which required a 90% CI for AUC between 0.80 to 1.25 and a 90% CI for 0.70 to 1.43. Methods for determining Cmax and AUC are well known in the art.

### VII. Examples

### General

Donepezil HCl polymorph Form I is characterized by an X-Ray Diffraction (XRD) Pattern having peaks at 9.9, 10.6, 12.7, 13.1, 13.7, 13.9, 14.9, 15.3, 16.1, 16.9, 17.5, 17.6, 18.4, 19.3, 19.8, 19.9, 21.2, 22.0, 22.5, 23.0, 23.6, 23.8, 23.9, 26.5, 28.0, and 29.5±0.2° 2θ. Donepezil HCl polymorph Form III is characterized by an X-Ray Diffraction (XRD) Pattern having peaks at 6.6, 9.9, 13.0, 15.0, 15.3, 15.7, 16.5, 17.4, 18.1, 18.5, 19.5, 20.1, 20.9, 21.7, 22.3, 22.9, 23.9, 24.7, 26.0, 27.2, 28.2, and 28.6±0.2° 2θ.

### Example 1. Preparation of Drug Matrix Layer with Donepezil HCl Polymorph Form III

An amount of 21.67 kg of triethyl citrate and 6.299 kg of lauryl lactate were mixed with 124.12 kg of ethyl acetate, and then 0.928 kg of ascorbyl palmitate was dissolved using a disperser. To the solution 27.79 kg of cross-linked polyvinyl pyrrolidone (Kollidon CL-M) was dispersed and homogenized. To the homogenized dispersion, 29.64 kg of donepezil hydrochloride polymorph Form III was added and mixed using an anchor, turbine and disperser agitation. The disperser was shut off and 3.705 kg of sorbitan monolaurate was then added and mixed using anchor and turbine agitation. The disperser was then re-started and 5.986 kg of sodium bicarbonate (with D90 particle size of 20 μm to 100 μm, 1.0:1.0 mole ratio to donepezil hydrochloride) and 22.15 kg of glycerin were added. Following this, the disperser is turned off again and 147.71 kg of acrylate copolymer (Duro-Tak 387-2287) was added to form the drug matrix wet adhesive formulation.

The drug matrix wet adhesive formulation was coated on a release liner and dried to get a dry coat weight of 120 g/m² to form a drug matrix dry adhesive formulation.

TABLE 1
Drug Matrix Layer Components

| | Drug | Drug | | Drug |
|---|---|---|---|---|
| | Matrix | Matrix | Drug | Matrix |
| | Wet | Dry | Matrix Dry | Dry |
| | Adhesive | Adhesive | Adhesive | Adhesive |
| | For- | For- | For- | For- |
| | mulation | mulation | mulation | mulation |
| Ingredient | (kg) | (kg) | (mol) | (% w/w) |
| Ethyl acetate | 124.12 | | | |
| Crospovidone, NF | 27.79 | 27.79 | | 14.42 |
| Ascorbyl Palmitate, NF | 0.928 | 0.928 | | 0.48 |
| Donepezil HCl, USP | 29.64 | 29.64 | 71.257 | 15.38 |
| Polymorph III | | | | |
| Triethyl Citrate, NF | 21.67 | 21.67 | | 11.24 |
| Lauryl Lactate | 6.299 | 6.298 | | 3.27 |
| Glycerin, USP | 22.15 | 22.15 | | 11.49 |
| Sorbitan Monolaurate, | 3.705 | 3.705 | | 1.92 |
| NF | | | | |
| Sodium bicarbonate, | 5.986 | 5.986 | 71.257 | 3.11 |
| with a D90 particle size | | | | |
| of 20 to 100 μm | | | | |
| Duro-Tak 387-2287 | 147.71 | 74.59 | | 38.70 |
| Total | 390 | 192.76 | | 100.00% |

### Example 2. Preparation of Drug Matrix Layer with Donepezil HCl Polymorph Form III

Additional drug matrix layers were prepared according to the method of Example 1 using a mole ratio of 0.88:1.0 or 0.92:1.0 sodium bicarbonate to donepezil HCl. Additional drug matrix layers can be prepared according to the method of Example 1 using a mole ratio of 0.95:1.0 sodium bicarbonate to donepezil HCl.

TABLE 2
Drug Matrix Layer Components

| | 0.88 molar | 0.92 molar | 0.95 Molar |
|---|---|---|---|
| | ratio | ratio | Ratio |
| | NaHCO₃ | NaHCO₃ | NaHCO₃ |
| | Drug Matrix | Drug Matrix | Drug Matrix |
| | Dry | Dry | Dry |
| | Adhesive | Adhesive | Adhesive |
| | Formulation | Formulation | Formulation |
| Ingredient | (% w/w) | (% w/w) | (%w/w) |
| Ethyl acetate | | | |
| Crospovidone, NF | 14.42 | 14.42 | 14.42 |
| Ascorbyl Palmitate, NF | 0.48 | 0.48 | 0.48 |
| Donepezil HCl, USP | 15.38 | 15.38 | 15.38 |
| Polymorph III | | | |
| Triethyl Citrate, NF | 11.24 | 11.24 | 11.24 |
| Lauryl Lactate | 3.27 | 3.27 | 3.27 |
| Glycerin, USP | 11.49 | 11.49 | 11.49 |
| Sorbitan Monolaurate, | 1.92 | 1.92 | 1.92 |
| NF | | | |
| Sodium bicarbonate, | 2.73 | 2.86 | 2.95 |
| with a D90 particle size | | | |
| of 20 to 100 μm | | | |
| Duro-Tak 387-2287 | 39.07 | 38.95 | 38.85 |
| Total | 100.00% | 100.00% | 100% |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of preparing a drug matrix layer, comprising:
forming a first mixture comprising donepezil HCl polymorph Form III, and sodium bicarbonate, thereby preparing the drug matrix layer.

2. The method of claim 1, wherein the donepezil HCl polymorph Form III is characterized by an X-Ray Diffraction (XRD) Pattern having peaks at 6.6, 15.0, 16.5, 18.5, 20.1, 21.7, 26.0, and 28.2±0.2° 2θ.

3. The method of claim 1 or 2, wherein the donepezil HCl polymorph Form III is further characterized by the XRD pattern having peaks at 18.1, 20.9, 22.3 and 27.2±0.2° 2θ.

4. The method of any one of claims 1 to 3, wherein the donepezil HCl polymorph Form III is further characterized by the XRD pattern having peaks at 9.9, 13.0, 15.3, 15.7, 17.4, 19.5, 22.9, 23.9, 24.7 and 28.6±0.2° 2θ.

5. The method of any one of claims 1 to 4, wherein the donepezil HCl polymorph Form III is characterized by the XRD pattern substantially as shown in FIG. 2.

6. The method of any one of claims 1 to 5, wherein the sodium bicarbonate is present in a molar ratio of at least 0.85 to the donepezil HCl.

7. The method of any one of claims 1 to 6, wherein the sodium bicarbonate is present in a molar ratio of at least 1.0 to the donepezil HCl.

8. The method of any one of claims 1 to 6, wherein the sodium bicarbonate is present in a molar ratio of 0.88 to the donepezil HCl.

9. The method of claim 8, comprising
forming the first mixture comprising donepezil HCl polymorph Form III, and sodium bicarbonate, wherein the sodium bicarbonate is present in a molar ratio of 0.88 to the donepezil HCl.

10. The method of any one of claims 1 to 6, wherein the sodium bicarbonate is present in a molar ratio of 0.92 to the donepezil HCl.

11. The method of claim 10, comprising
forming the first mixture comprising donepezil HCl polymorph Form III, and sodium bicarbonate, wherein the sodium bicarbonate is present in a molar ratio of 0.92 to the donepezil HCl.

12. The method of any one of claims 1 to 7, wherein the sodium bicarbonate is present in a molar ratio of 1.0 to the donepezil HCl.

13. The method of claim 12, comprising
forming the first mixture comprising donepezil HCl polymorph Form III, and sodium bicarbonate, wherein the sodium bicarbonate is present in a molar ratio of 1.0 to the donepezil HCl.

14. The method of any one of claims 1 to 13, further comprising
adding triethyl citrate, lauryl lactate, and ethyl acetate to the first mixture;
adding ascorbyl palmitate to the first mixture;
adding polyvinylpyrrolidone to the first mixture;
adding sorbitan monolaurate to the first mixture; and
adding an acrylate polymer to the first mixture, thereby preparing the drug matrix layer.

15. The method of claim 14, further comprising:
coating the first mixture on a release liner; and
drying the coated mixture.

16. A drug matrix layer prepared by the method of any one of claims 1 to 15.

17. A method for preparing a transdermal delivery system, comprising:
(i) laminating a microporous membrane layer onto a top surface of a contact adhesive layer to form a contact adhesive laminate having a top surface and a bottom 3 surface;
(ii) preparing a drug matrix layer comprising:
forming a first mixture comprising triethyl citrate, lauryl lactate, and ethyl acetate,
adding ascorbyl palmitate to the first mixture,
adding polyvinylpyrrolidone to the first mixture,
adding donepezil HCl polymorph Form III to the first mixture,
adding sorbitan monolaurate to the first mixture,
adding sodium bicarbonate and glycerin to the first mixture, wherein the sodium bicarbonate is present in a molar ratio of at least 0.85 to the donepezil HCl,
adding an acrylate polymer to the first mixture,
coating the first mixture on a release liner,
drying the coated mixture, and
removing the release liner, thereby preparing the drug matrix layer;
(iii) laminating the drug matrix layer onto the top surface of the contact adhesive laminate to form a drug matrix laminate having a top surface and a bottom surface;
(iv) laminating a separating layer onto the top surface of the drug matrix laminate to form an active laminate having a top surface and a bottom surface, wherein the separating layer comprises a top surface and a bottom surface, wherein the top surface of the separating layer comprises a coating of ethylene-vinyl acetate copolymer, and wherein the bottom surface of the separating layer is in contact with the top surface of the drug matrix laminate;
(v) laminating a polyester fabric onto an adhesive overlay layer comprising acrylate polymer to form a backing layer having a top surface and a bottom surface;
(vi) laminating the bottom surface of the backing layer onto the top surface of the active laminate so that the adhesive overlay layer is in contact with the top surface of the active laminate;
(vii) treating the top surface of the separating layer with a corona discharge treatment to form a treated separating layer,
wherein the corona discharge treatment is performed using a power of from 0.10 KW to 0.12 kW and a power density of from 2.1 to 2.6 W/ft²/min,
wherein the treated separating layer comprises a top surface and a bottom surface such that the top surface of the treated separating layer has a surface energy of at least 40 Dynes, and
wherein the bottom surface of the contact adhesive layer is in contact with a first process liner;
(viii) removing the first process liner to expose the bottom surface of the contact adhesive layer; and
(ix) laminating a release liner onto the bottom surface of the contact adhesive layer, thereby forming the transdermal delivery system.

18. A transdermal delivery system prepared by the method of claim 17.
